(19) United States
(12) Patent Application Publication
(10) Pub. No.: US 2025/0032200 A1
(43) Pub. Date: Jan. 30, 2025

(54) ROTATABLE MOTOR WITH AMBIENT MAGNETIC FIELD STATOR

(71) Applicant: 4D SURGICAL, INC., Salt Lake City, UT (US)

(72) Inventor: Lorne Wyatt Hofstetter, Salt Lake City, UT (US)

(73) Assignee: 4D SURGICAL, INC., Salt Lake City, UT (US)

(21) Appl. No.: 18/716,624

(22) PCT Filed: Dec. 7, 2022
(86) PCT No.: PCT/US22/81116

§ 371 Date: Jun. 5, 2024

Related U.S. Application Data
(60) Provisional application No. 63/265,053, filed on Dec. 7, 2021.
(60) Provisional application No. 63/386,100, filed on Dec. 5, 2022.

(51) Int. Cl. A61B 34/30; A61B 5/00; A61B 5/055; A61B 34/20; B25J 9/12

(52) U.S. Cl. CPC A61B 34/30; A61B 5/0036; A61B 5/055; B25J 9/126; A61B 2034/2059

(57) ABSTRACT

Apparatus and associated methods relate to an ambient magnetic field motor (AMFM) operable in a strong ambient magnetic (e.g., electromagnetic) field (AMF) having at least one rotor, each with respective shafts extending a corresponding rotation axis. The motor may, for example, be activated in response to the AMF to generate an output torque. In an illustrative example, the motor may, for example, include a mechanical output shaft mechanically coupled to the at least one rotor. For example, the mechanical output shaft may, for example, extend along a longitudinal axis intersecting at least one corresponding rotation axis of the at least one rotor. For example, in an AMF, the at least one rotor may, for example, be induced to rotate by the AMF in response to a selectively applied electrical current to generate an output torque at the mechanical output shaft about the longitudinal axis. Various embodiments may, for example, advantageously provide an electromagnetic motor operable in a strong AMF.

### CROSS-REFERENCE TO RELATED APPLICATIONS

This application claims the benefit of U.S. Provisional Application Ser. No. 63/265,053, titled “Rotatable Motor with Ambient Magnetic Field Stator,” filed by Lorne Wyatt Hofstetter, on Dec. 7, 2021. This application claims the benefit of U.S. Provisional Application Ser. No. 63/386,100, titled “Articles Evidencing Long-Felt Unmet Need and Failure by Others and Industry Skepticism,” filed by Lorne Wyatt Hofstetter, on Dec. 5, 2022.

This application incorporates the entire contents of the foregoing application(s) herein by reference.

### TECHNICAL FIELD

Various embodiments relate generally to electromagnetic motors.

### BACKGROUND

Robotic surgeries are surgical procedures that are done using robotic systems. For example, in a robotic assisted minimally invasive procedure, a surgeon may, in some cases, remotely control the robotic system using a remote manipulator. The surgeon may perform normal movements associated with the procedure using, for example, a robotic arm. The robotic arm may carry out these movements using end-effectors and manipulators. In a computer-controlled robotic system, the surgeon may use a computer to control the robotic arm and the end-effectors.

Magnetic resonance imaging (MRI) is a medical imaging technique that uses a magnetic field and computer-generated radio waves to create detailed images of the organs and tissues in a body. In various examples, MRI machines are large, tube-shaped magnets. When a patient lies inside an MRI machine, an ambient magnetic field temporarily realigns water molecules in a body to cause these aligned atoms (in the water molecules) to produce faint signals. For example, a computer device may generate images based on the signals. MRI may, for example, be especially helpful for imaging the brain, nerves, pathologic tissue, and/or various soft tissues.

Intraoperative magnetic resonance imaging may, for example, refer to an operating room configuration that includes imaging a patient via an MRI scanner while the patient is undergoing a surgery (e.g., brain surgery). In some cases, Neurosurgeons may use intraoperative MRI technology to obtain accurate pictures of the brain to guide them in removing brain tumors and treating other conditions such as epilepsy. In various examples, intraoperative MRI reduces the risk of damaging critical parts of the brain. For example, a neurosurgeon may use images from an intraoperative MRI scanner to determine whether additional resection is needed before the patient's head is closed and the surgery completed. In additional examples, intraoperative images may be used to guide minimally invasive procedures that utilize small incision sites such as brain biopsy, targeted drug delivery, biopsy of spinal lesions, joint repair, ligament repair, tendon repair, deep brain stimulator lead placement, and laser interstitial thermal therapy, among others.

### SUMMARY

Apparatus and associated methods relate to an ambient magnetic field motor (AMFM) operable in a strong ambient magnetic (e.g., electromagnetic) field (AMF) having at least one rotor, each with respective shafts extending a corresponding rotation axis. The motor may be activated in response to the AMF to generate an output torque. In an illustrative example, the motor may include a mechanical output shaft mechanically coupled to the at least one rotor. For example, the mechanical output shaft may extend along a longitudinal axis intersecting at least one corresponding rotation axis of the at least one rotor. For example, in an AMF, the at least one rotor may be induced to rotate by the AMF in response to a selectively applied electrical current to generate an output torque at the mechanical output shaft about the longitudinal axis. Various embodiments may advantageously provide an electromagnetic motor operable in a strong AMF.

Apparatus and associated methods relate to an ambient magnetic field motor (AMFM) operable in a strong ambient magnetic (e.g., electromagnetic) field (AMF) having at least two rotors. In an illustrative example, each rotor may be configured to rotate about a corresponding rotation axis based on a direction vector of an AMF. For example, a mechanical output shaft (MOS), mechanically coupled to the at least two rotors, may extend along a longitudinal axis intersecting at least one of the corresponding rotation axes of the at least two rotors. For example, in an AMF, rotation of the at least two rotors may be induced by the AMF in response to a selectively applied electrical current to generate a combined output torque at the MOS about the longitudinal axis. Various embodiments may advantageously provide a torque constant (k_{T}) at the MOS maintained within a predetermined range when an orientation of the AMFM changes.

Apparatus and associated methods relate to a hybrid AMFM. In an illustrative example, the AMFM may include a rotor and an auxiliary stator (AUXSTAT). The rotor, for example, may be configured to rotate about a rotational axis when induced by a magnetic field. The AUXSTAT may be configured to selectively generate an auxiliary magnetic field (AUXMF). A mechanical output shaft (MOS) may, for example, be mechanically coupled to the rotor. For example, in an ambient magnetic (e.g., electromagnetic) field (AMF), the rotor may be induced by the AMF and electric current through at least one rotor to generate an output torque at the MOS. When the output torque is below a predetermined threshold, the AUXSTAT may be activated to generate the AUXMF to induce rotation at the rotor by the AUXMF and a selectively applied electrical current. Various embodiments may advantageously provide a hybrid motor operable independent of an AMF.

Various embodiments may achieve one or more advantages. For example, some embodiments may combine rotation torque generated from two independently induced rotors to generate a single output torque along a third axis. Some embodiments, for example, may include more than two rotors to advantageously reduce variation in the torque constant when the orientation of the AMFM changes. For example, some embodiments may apply electromagnetic actuation principles to build multi-degree of freedom robotic systems that can safely operate in regions containing a strong magnetic field. Various embodiments may simplify development and construction of magnetic resonance imaging (MRI)-guided robotic systems for medical applications. Various embodiments may advantageously reduce cost and/or increase functionality in MRI-compatible robotic systems. Various embodiments may, for example, advantageously enable diagnostic applications that require an actuator(s) near the patient. For example, some embodiments may advantageously provide actuators for applications including mechanical excitation of tissues to enable quantification of tissue stiffness with MRI.

The details of various embodiments are set forth in the accompanying drawings and the description below. Other features and advantages will be apparent from the description and drawings, and from the claims.

### BRIEF DESCRIPTION OF THE DRAWINGS

FIG. 1A depicts an exemplary ambient magnetic field motor (AMFM) employed in an illustrative use-case scenario.

FIG. 1B is a block diagram depicting an exemplary AMFM system.

FIG. 2A and FIG. 2B depict a perspective view and a top view, respectively, of an exemplary AMFM.

FIG. 3A and FIG. 3B depict an exemplary AMFM including coupling gears for coupling of mechanical output from each rotor.

FIG. 4A, FIG. 4B, FIG. 4C, FIG. 4D, FIG. 4E, FIG. 4F, and FIG. 4G depict operations of an exemplary AMFM in exemplary orientations relative to an ambient magnetic field.

FIG. 5A and FIG. 5B depict an exemplary 3-rotor AMFM.

FIG. 6 depicts an exemplary result of a theoretical torque constant (k_{T}) for a 1-rotor motor, a 2-rotor motor, and a 3-rotor motor.

FIG. 7A depicts an exemplary brush, rotor with armature winding, and mechanical commutator.

FIG. 7B shows an exemplary mechanical commutation scheme for a rotor armature having three rotating coils that are separated by 120°.

FIG. 8A shows an exemplary two-rotor AMFM with two Hall effect sensors mounted on a motor housing.

FIG. 8B shows an exemplary circuit diagram for switching current based on readings from the Hall effect sensors in the AMFM as described with reference to FIG. 8A.

FIG. 8C depicts an exemplary logic table with illustrative switching logic for the exemplary AMFM as shown in FIGS. 8A-8B.

FIG. 9A depicts a schematic of an exemplary 8-conductor slip ring assembly

FIG. 9B depicts an exemplary rotor, rotor axle, and power transfer device for the 8-conductor slip ring AMFM system as described with reference to FIG. 9A.

FIG. 10A depicts a block diagram of an exemplary AMFM system.

FIG. 10B shows an exemplary driver circuit of the exemplary AMFM system as described with reference to FIG. 10A.

FIG. 11A and FIG. 11B depict an exemplary embodiment with a single rotor motor.

FIG. 12 depicts a schematic diagram of an exemplary AMFM closed loop control system.

FIG. 13 depicts an exemplary motor controller, switching circuitry, power supply inverters, and associated hardware enclosed in a conducting enclosure.

FIG. 14A, FIG. 14B, and FIG. 14C depict an exemplary implementation of an AMFM in the patient area of an MRI scanner.

FIG. 15A and FIG. 15B depict an exemplary hybrid AMFM.

FIG. 16A and FIG. 16B depict an exemplary hybrid AMFM with a coreless rotor.

FIG. 17 depicts an exemplary hybrid AMFM system.

Like reference symbols in the various drawings indicate like elements.

### DETAILED DESCRIPTION OF ILLUSTRATIVE EMBODIMENTS

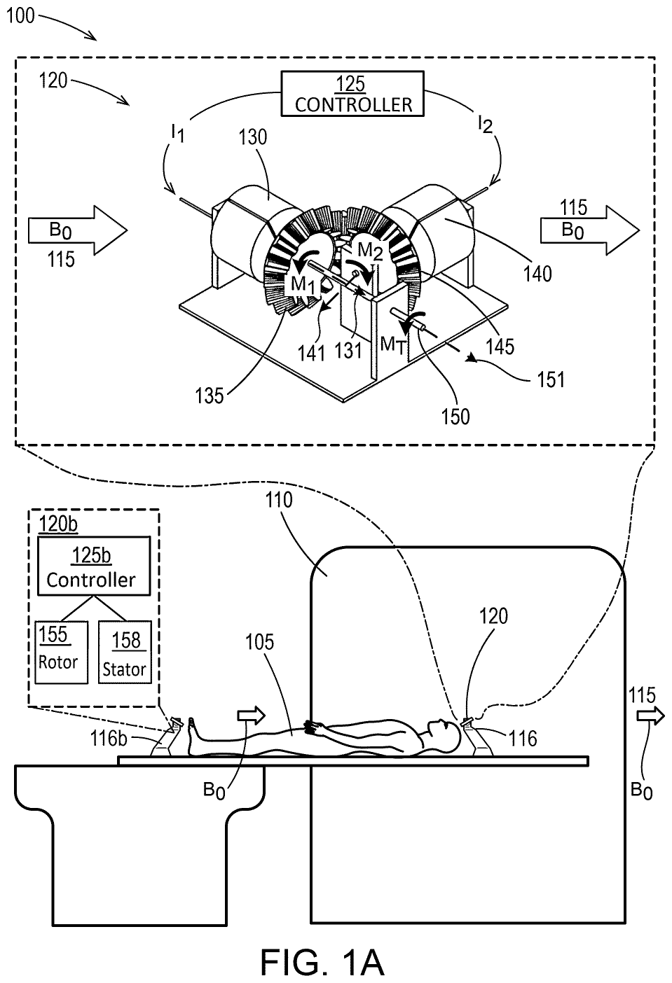

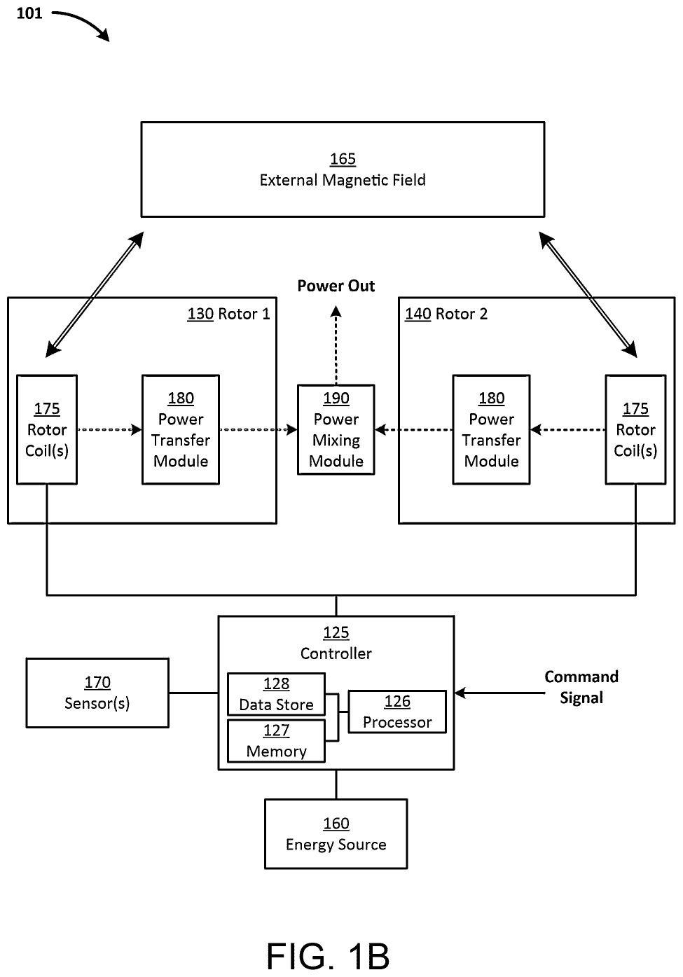

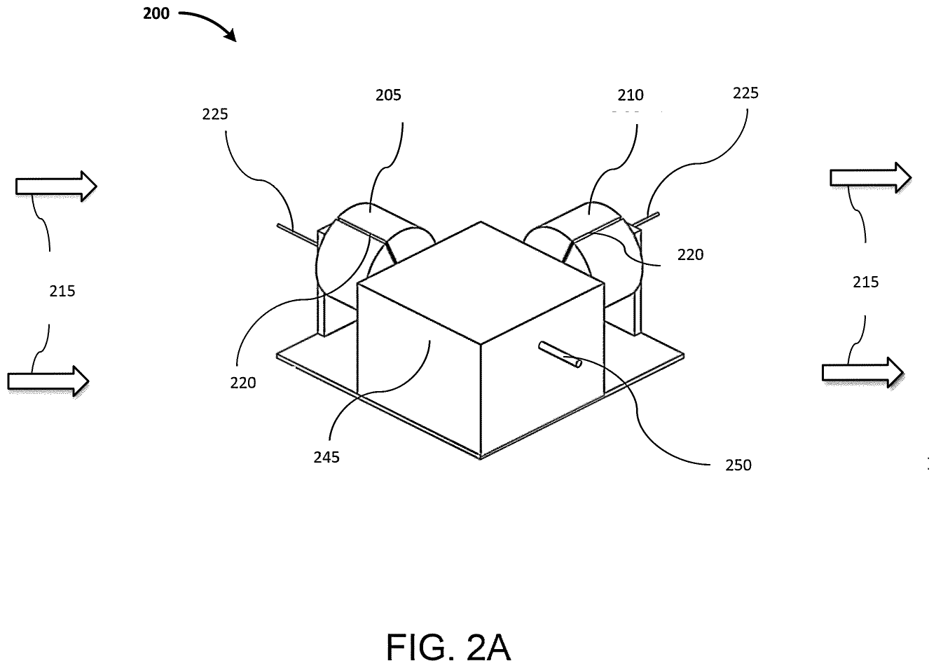

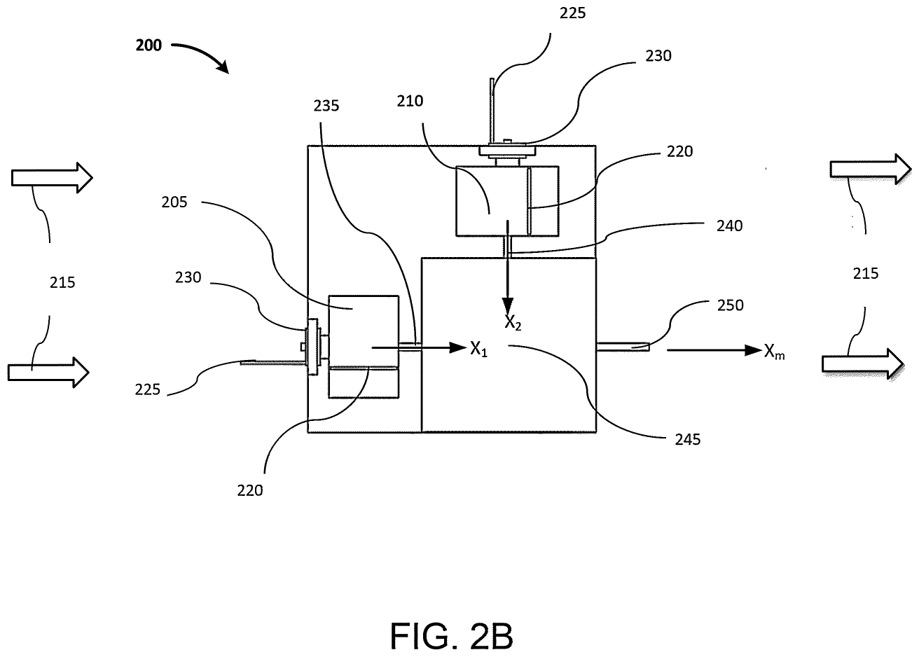

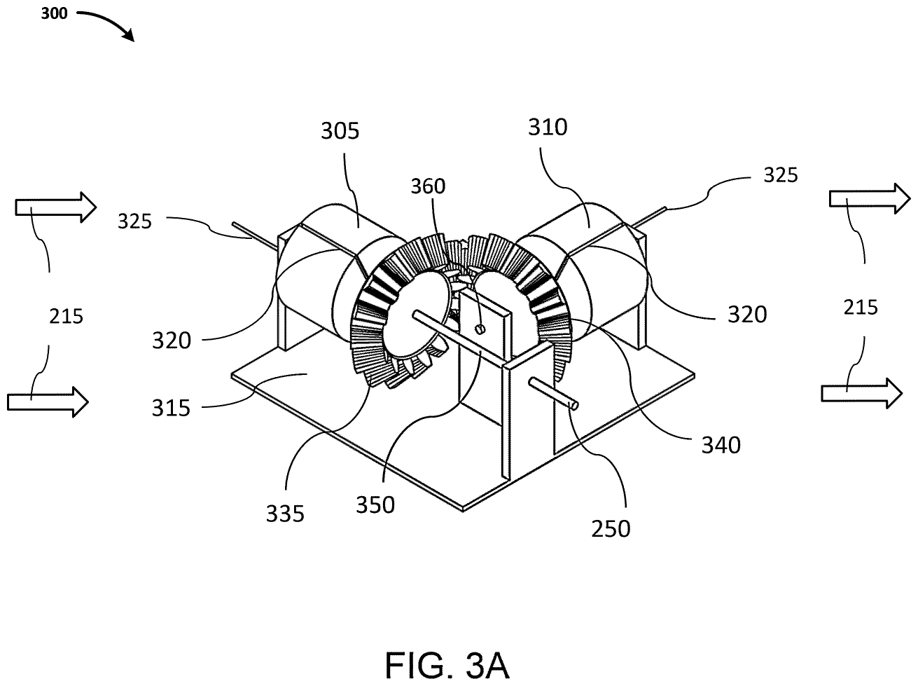

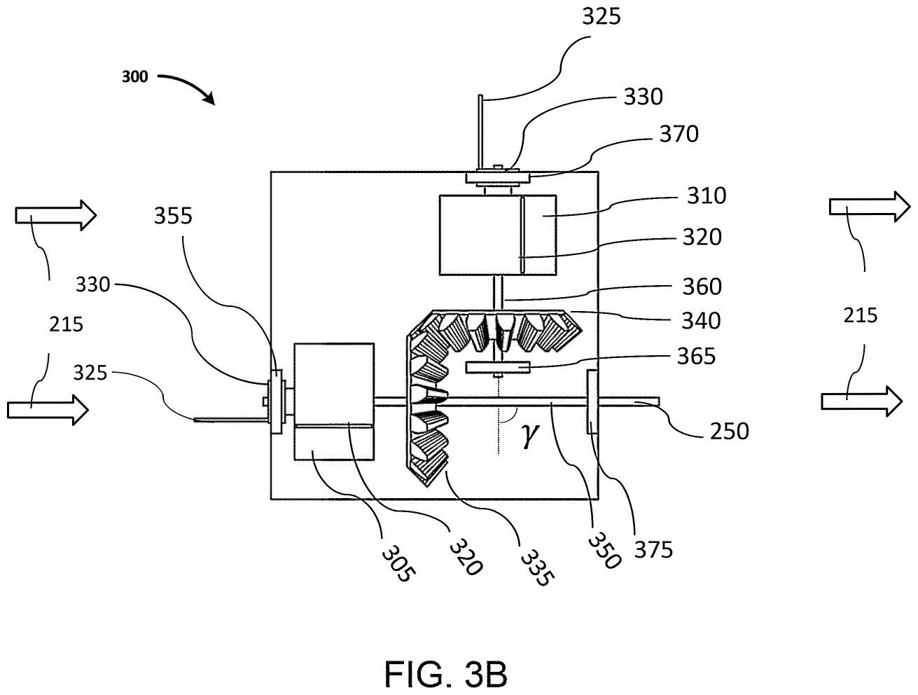

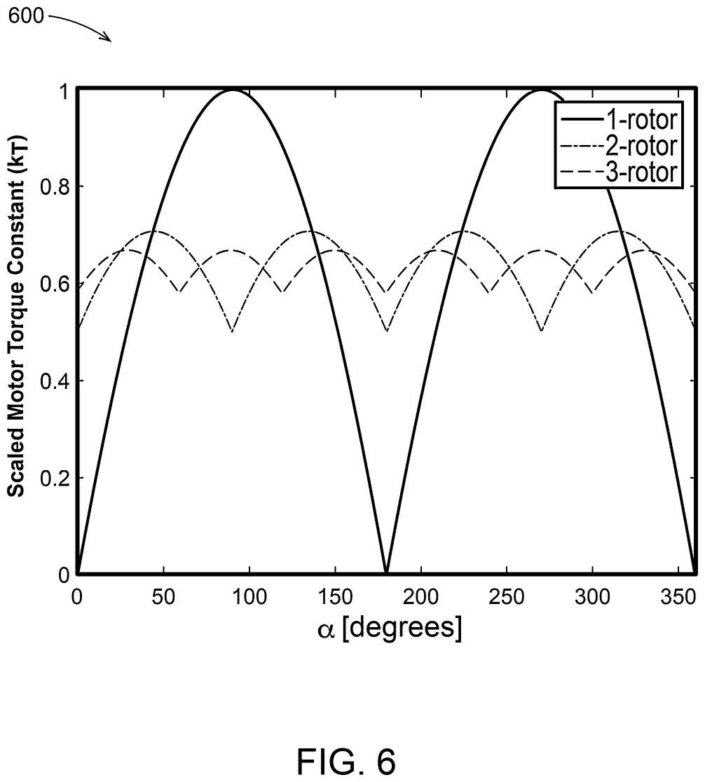

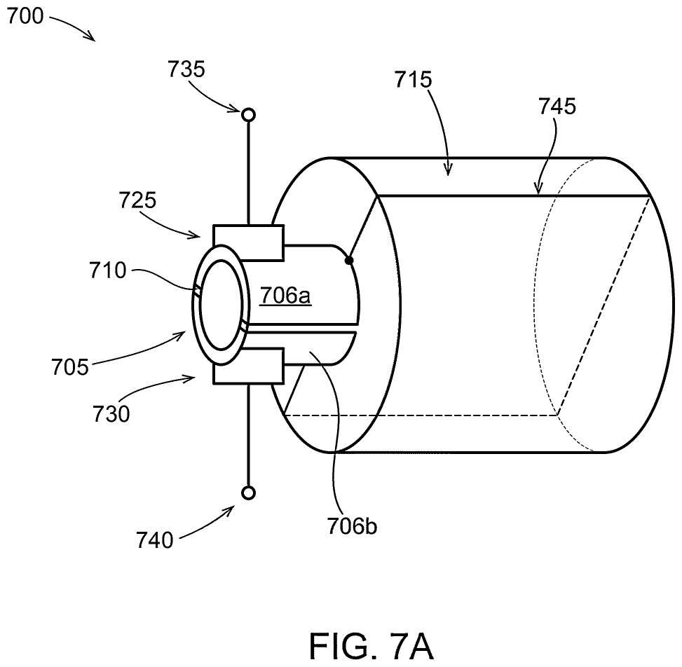

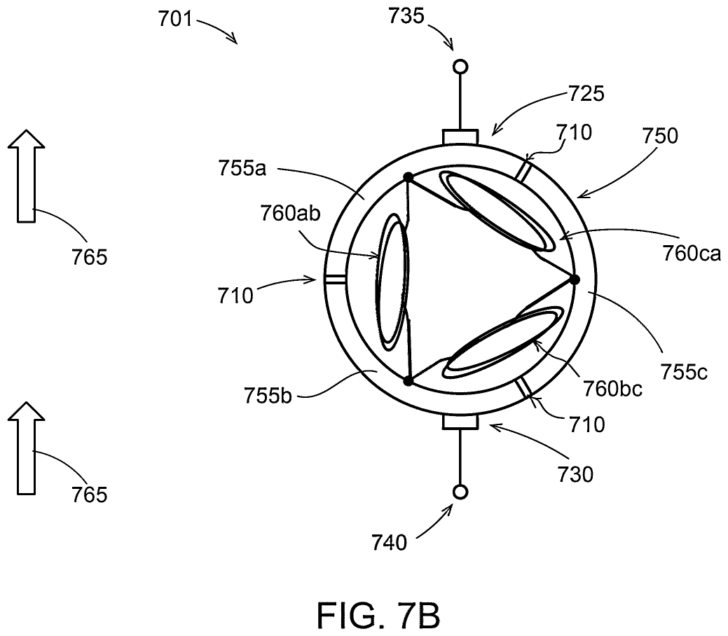

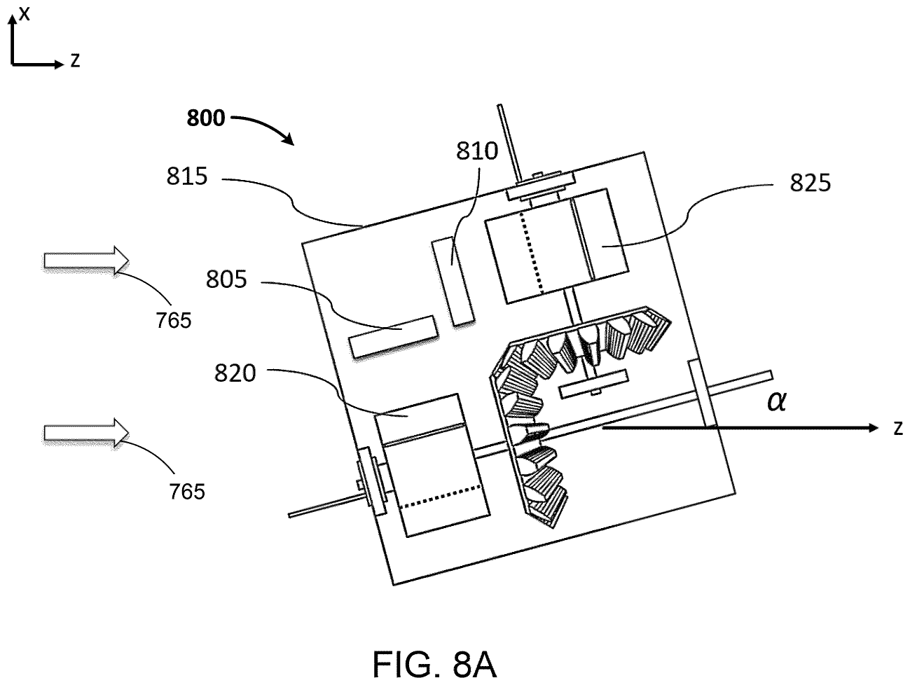

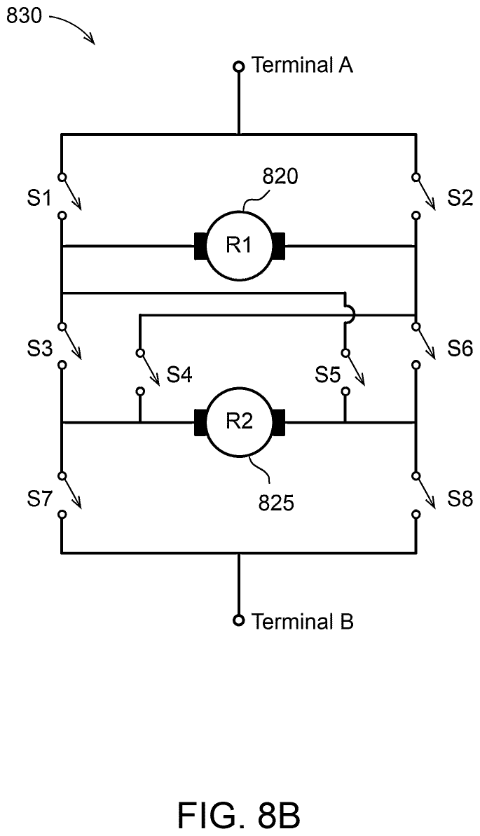

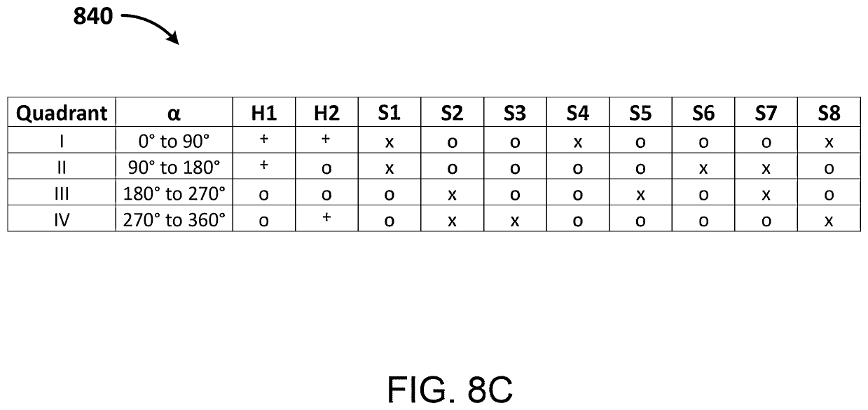

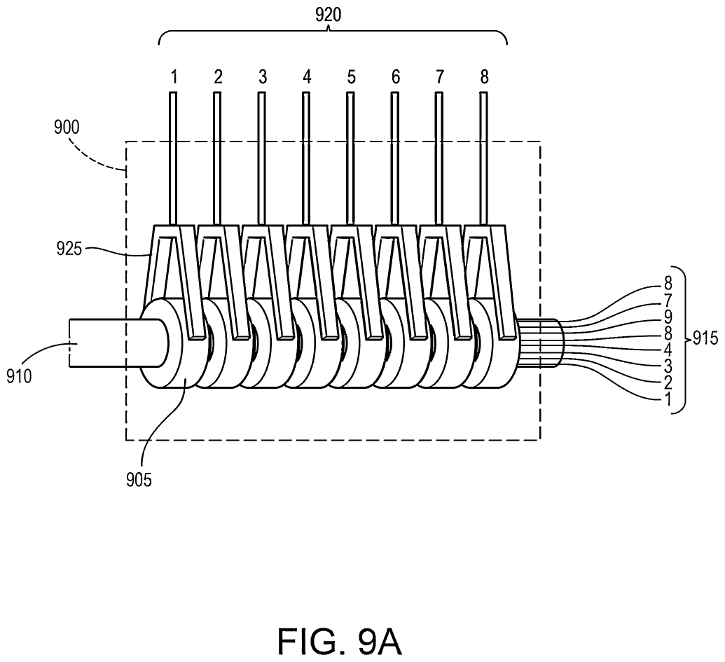

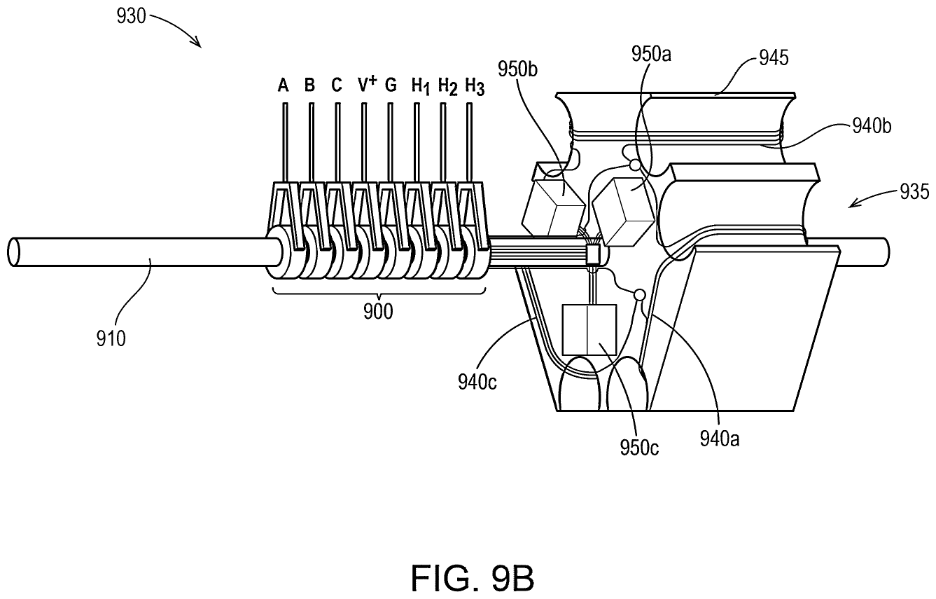

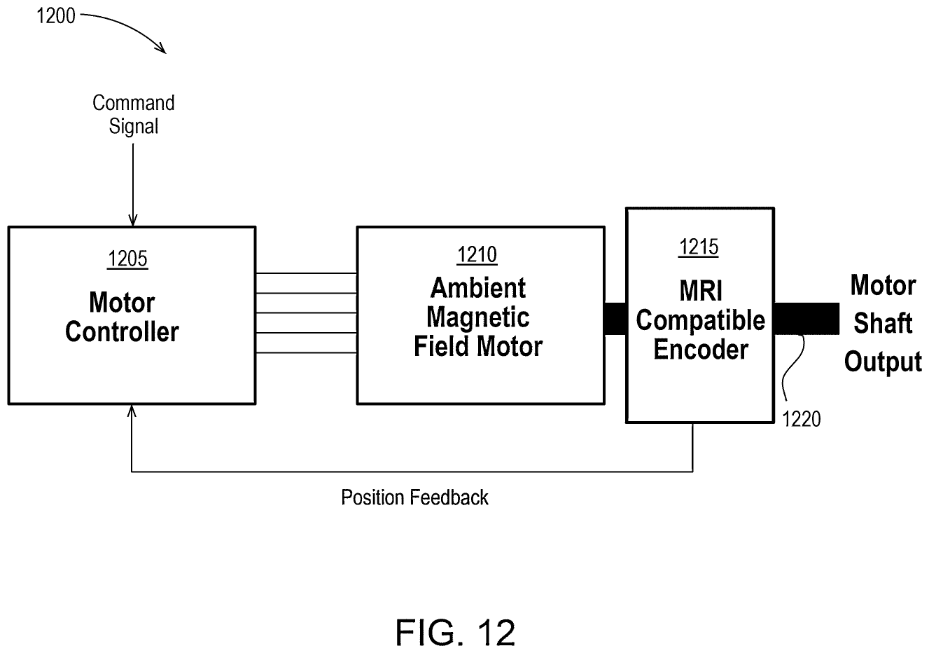

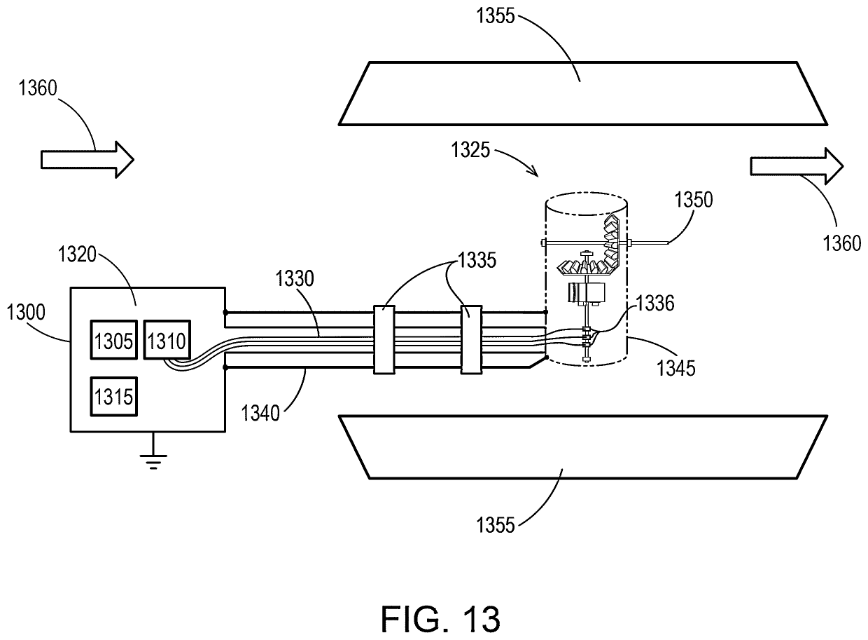

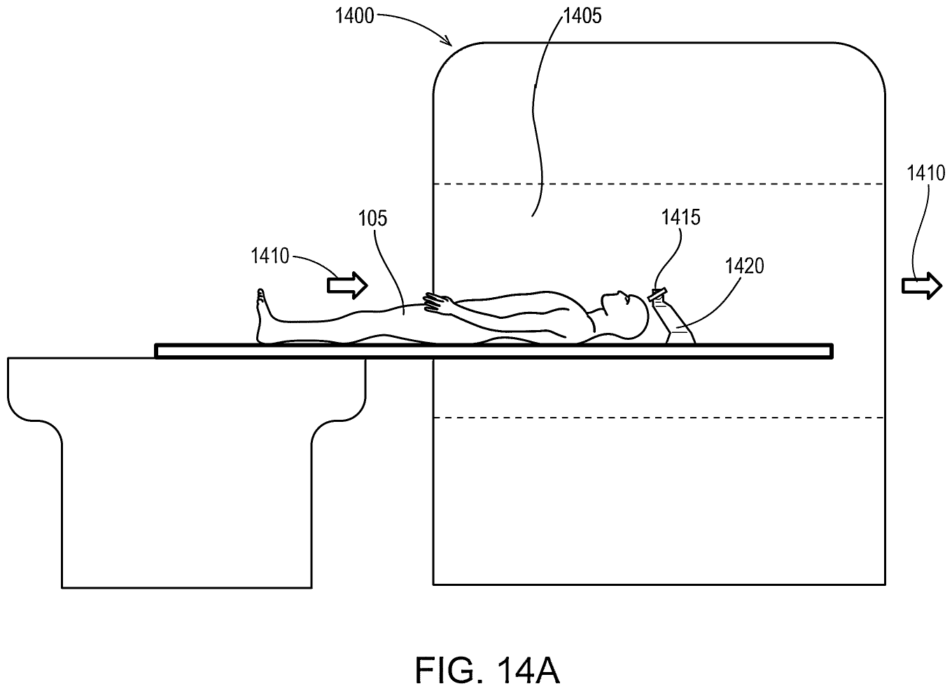

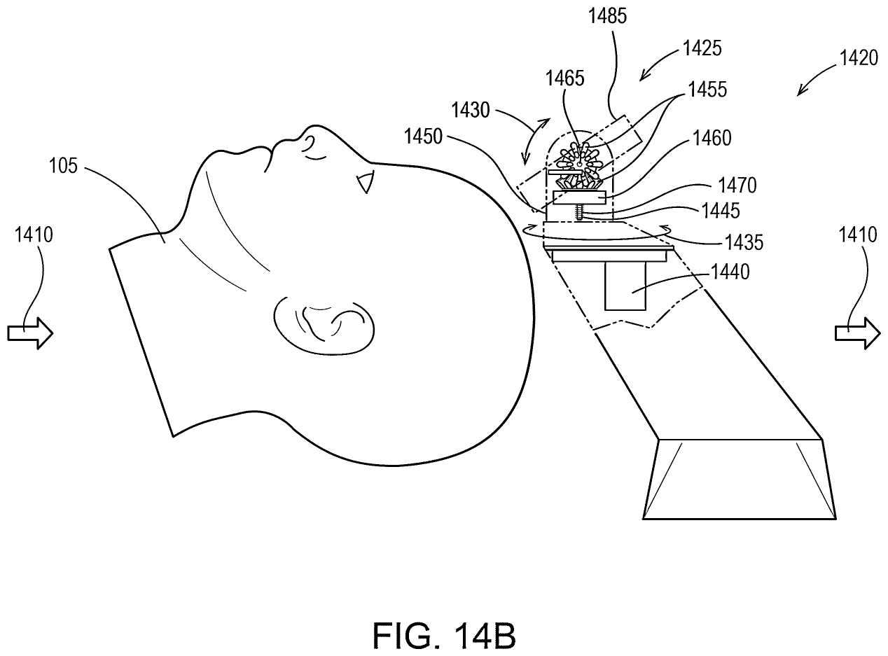

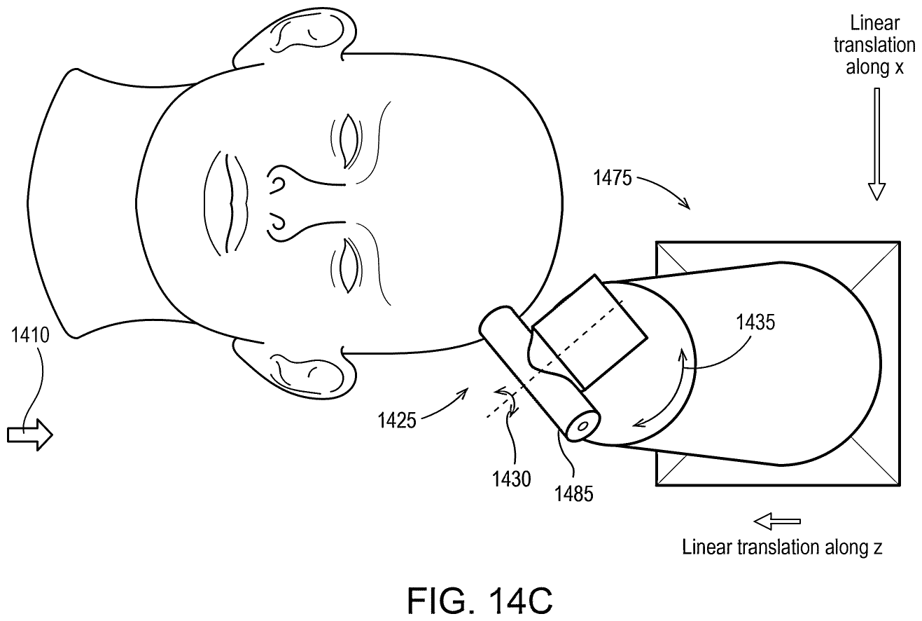

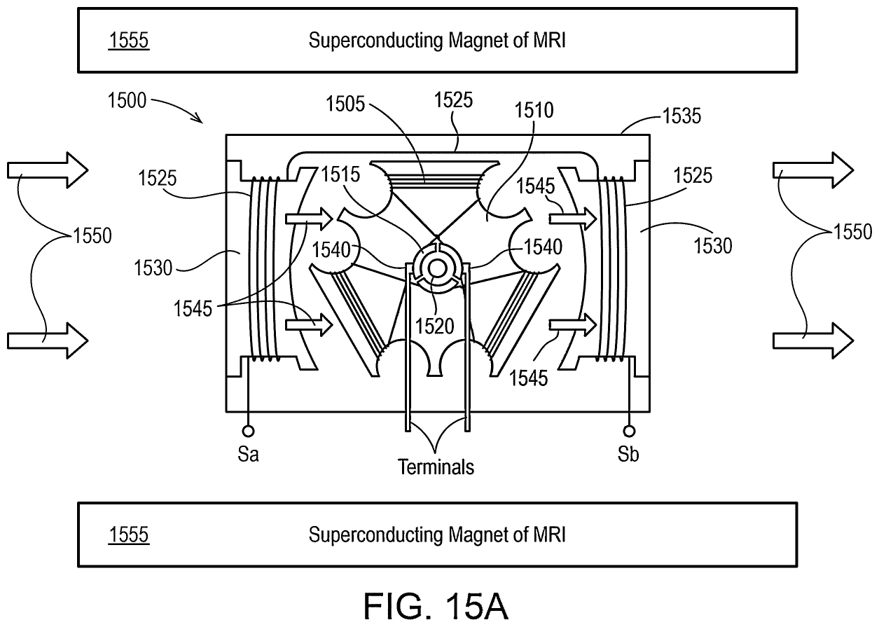

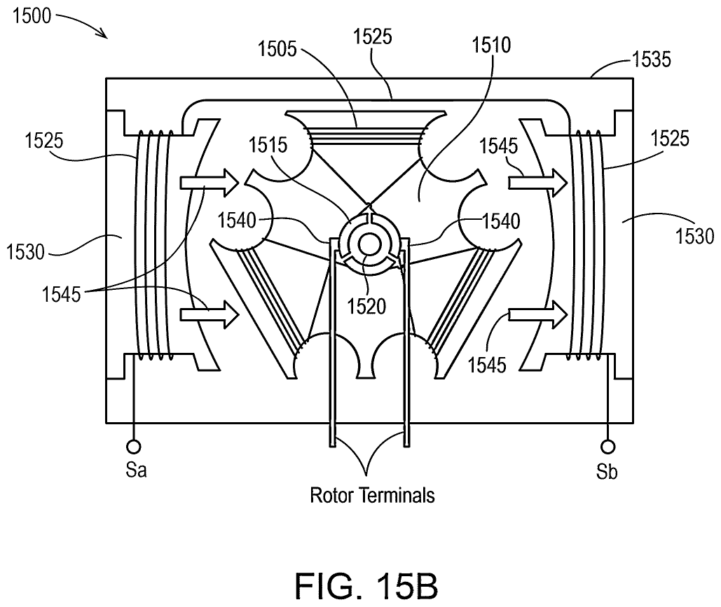

To aid understanding, this document is organized as follows. First, to help introduce discussion of various embodiments, an ambient magnetic field motor (AMFM) system is introduced with reference to FIGS. 1A-2B. Second, that introduction leads into a description with reference to FIGS. 3A-5B of exemplary AMFM embodiments and exemplary AMFM operation. Third, with reference to FIG. 6, a comparison of exemplary AMFM embodiments is discussed. Fourth, with reference to FIGS. 7A-7B, exemplary AMFM embodiments related to electrical power transfer to the rotor with a mechanical commutator are discussed. Fifth, with reference to FIGS. 8A-8C, the discussion turns to exemplary embodiments that illustrate AMFM embodiments with exemplary feedback and/or control. Sixth, and with reference to FIGS. 9A-11B, this document describes exemplary AMFM systems implemented using slip rings for electric power transfer to the rotor and switching circuitry for electrical commutation. Seventh, this disclosure turns to a discussion of position feedback and RF shielding with reference to FIGS. 12-13. Illustrative implementations are discussed with respect to FIGS. 14A-14C. Eighth, with reference to FIGS. 15A-17, exemplary embodiments of MRI-compatible hybrid AMFM systems are described. Finally, the document discusses further embodiments, exemplary applications and aspects relating to AMFM systems.

FIG. 1A depicts an exemplary ambient magnetic field motor (AMFM) employed in an illustrative use-case scenario. In an exemplary scenario 100, a patient 105 is positioned partially within an MRI scanner 110. The MRI scanner 110 generates an ambient field B₀ 115. The robotic surgical system (RSS) 116 is provided with an AMFM 120 to operate an end effector of the RSS 116 (e.g., a surgical tool).

In the depicted example, the AMFM 120 includes a controller 125. The controller 125 controls a first current (11) in one or more coils of a first rotor 130. The first rotor 130 generates an output moment M₁ (e.g., on an output shaft) about a first axis 131 in response to B₀ and I₁. The first rotor 130 is coupled to a first gear 135 to apply the M₁ to the first gear 135.

The controller 125 controls a second current (I₂) in one or more coils of a second rotor 140. The second rotor 140 generates an output moment M₂ about a second axis 141 in response to the B₀ and the I₂. The second rotor 140 is coupled to a second gear 145 to apply the M₂ to the second gear 145.

In various examples, B₀ may represent a strength of the ambient magnetic field. For example, the strength may be an intensity vector that includes a magnitude of the field and a direction (orientation) of the field. A RSS 116 is positioned in the MRI scanner 110 to operate on a head of the patient 105. In some examples, the output moments M₁ and M₂ generated by the first rotor 130 at current I₁ and the second rotor 140 at current I₂ may depend on a position and/or orientation of the RSS 116 within the MRI scanner 110.

The first gear 135 and the second gear 145 are configured to cooperate to combine the M₁ and the M₂ to generate a combined output moment Mr about an output shaft 150 extending along a longitudinal axis 151. The controller 125 may control the I₁ and the I₂ such that M₁ and M₂ sum, via (mechanical) interaction of the first gear 135 and the second gear 145, to generate a desired Mr. Accordingly, as the AMFM 120 changes in orientation relative to the B₀, the first rotor 130 and the second rotor 140 may be selectively controlled to maintain a desired output moment on the output shaft 150.

Such embodiments may, for example, advantageously enable the elimination of ferrous materials. For example, some embodiments may advantageously include at least two non-ferrous rotors configured to rotate along non-parallel axes in the presence of an ambient magnetic field (e.g., serving as a stator field).

Various embodiments may advantageously enable deployment of an electromagnetic motor and/or electromagnetic motor controllers (e.g., including proportional-integral-derivative (PID) control schemes) in a strong ambient magnetic field (e.g., within an MRI scanner). For example, various such embodiments may advantageously operate in an ambient magnetic field of at least 0.1 Tesla (T). Some embodiments may advantageously operate in an ambient magnetic field of at least 1.5T. Some embodiments may advantageously operate in an ambient magnetic field of 3T. Some embodiments may advantageously operate in an ambient magnetic field of 7T.

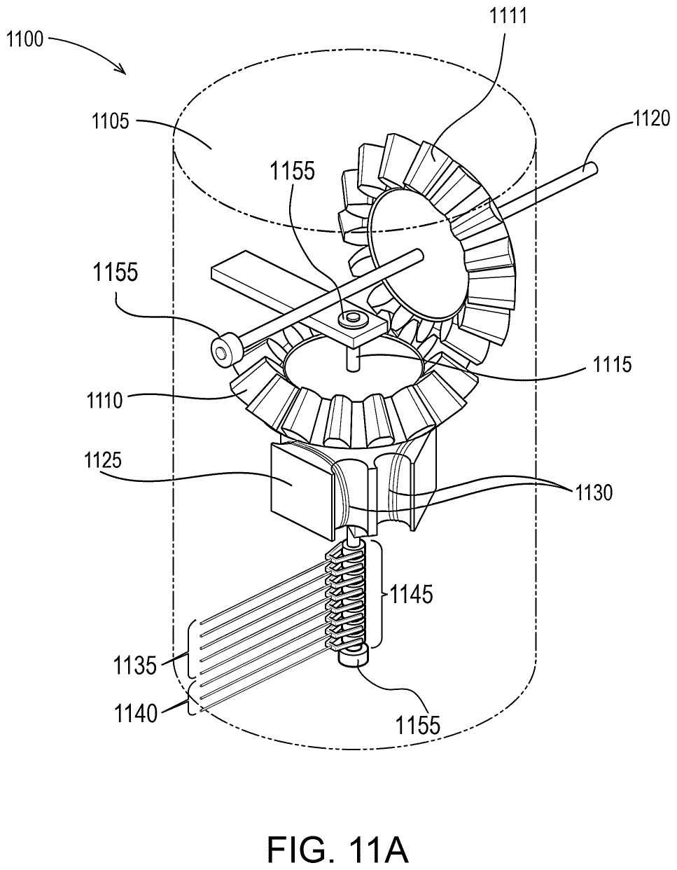

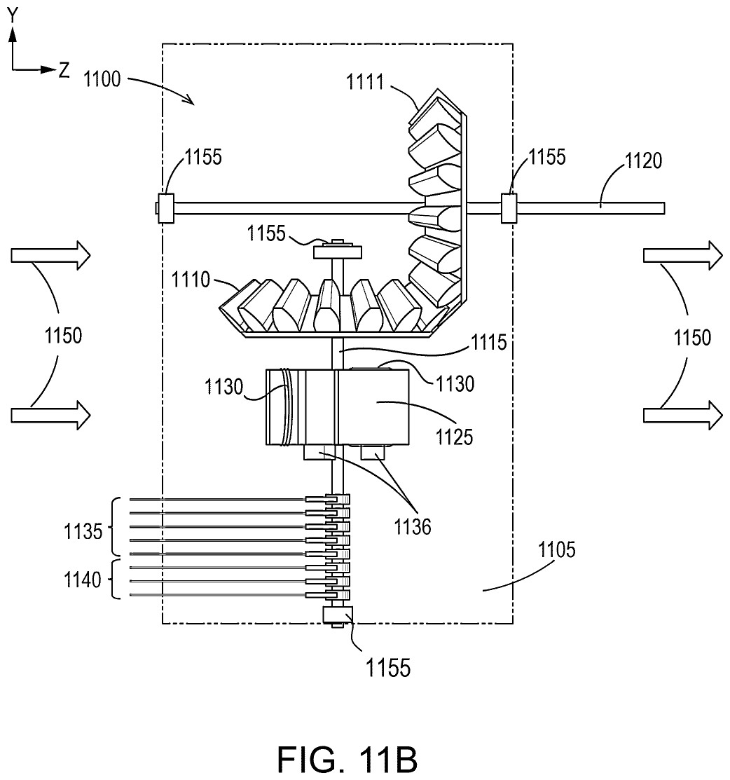

Although the AMFM 120 is depicted as a dual-rotor controller, various embodiments (e.g., as disclosed at least with reference to FIGS. 11A-11B) may be configured, by way of example and not limitation, with a single rotor having an output shaft angled relative to an axis parallel to the motor output shaft. Some such embodiments may, for example, include a slip ring power transfer device. Various such embodiments may, for example, advantageously provide a single-rotor AMFM maneuverable in an ambient magnetic field. Some embodiments (e.g., as disclosed at least with reference to FIGS. 15A-17, may, for example, include an auxiliary stator configured to selectively produce a stator field in location (e.g., to advantageously enable actuation where the ambient magnetic field is weak and/or incorrectly aligned with the rotor axis).

As shown in FIG. 1A, a second RSS 116b is provided with a second AMFM 120b to operate an end effector of the second RSS 116b. The second RSS 116b is, in this example, located outside of the MRI scanner 110. For example, the second AMFM 120b may be under a weak or without an ambient magnetic field (e.g., at a foot of the patient 105 where the external magnetic field 165 may be weak).

In the depicted example, the hybrid stator AMFM 120b includes a controller 125b, a rotor 155 (e.g., configured such as the rotor 130 and/or the rotor 140) and an auxiliary stator 158. In some implementations, the controller 125b may deliver an appropriate electrical current to the rotor 155 and/or the stator 158 of the hybrid stator AMFM 120b to achieve a desired mechanical output. In some implementations, the auxiliary stator 158 may be controlled to produce a stator field that produces a controlled torque output even when the ambient magnetic field direction of B₀ is not appropriate for actuation.

Accordingly, for example, the RSS 116b may be configured to be operable both within and outside of the MRI scanner 110. For example, the hybrid stator AMFM 120b may be configured to be MRI compatible. For example, the hybrid stator AMFM 120b may be operable also without ambient magnetic field. In various implementations, the hybrid stator AMFM 120b may advantageously allow the RSS 116b to operate in locations where the ambient magnetic field is weak.

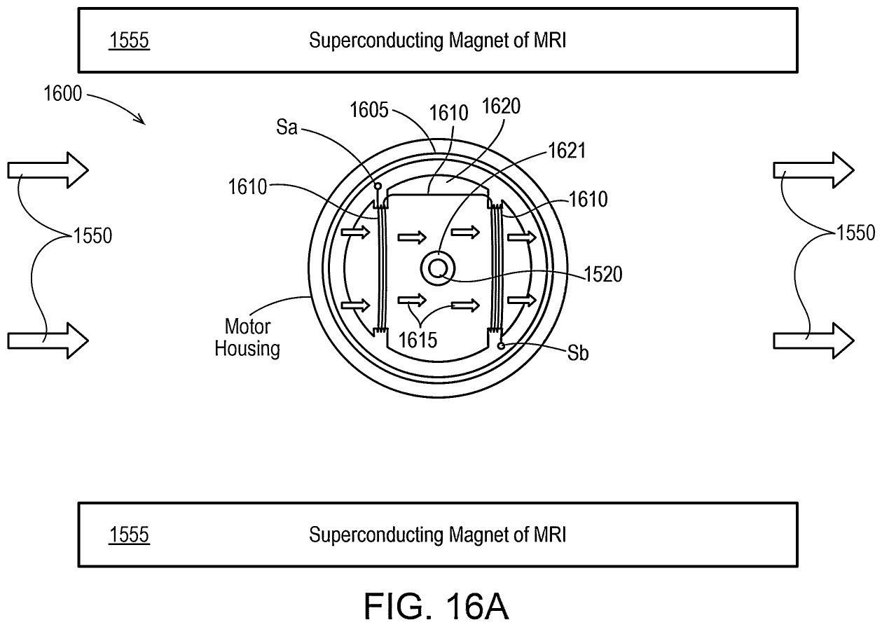

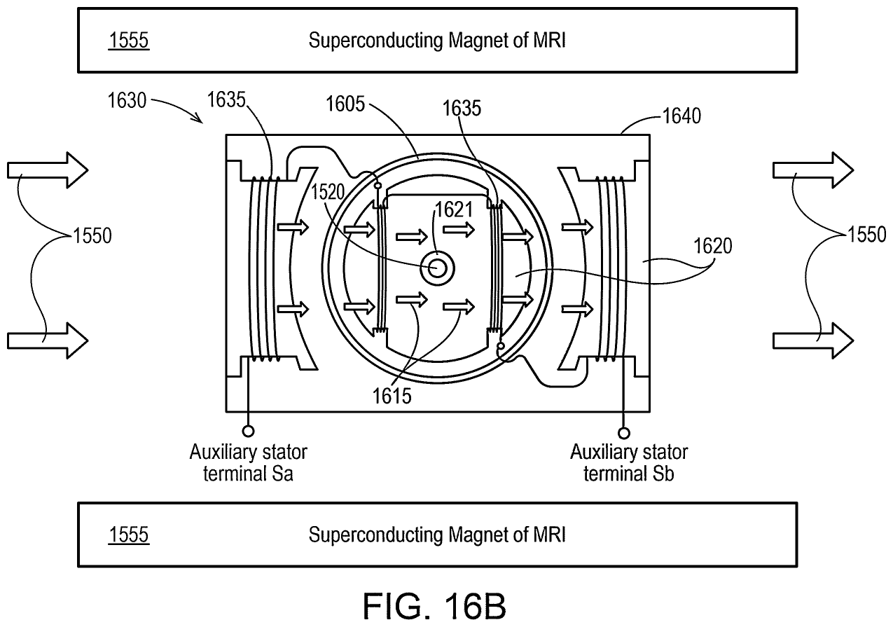

Some embodiments of the hybrid stator AMFM 120b (e.g., as disclosed at least with reference to FIGS. 15A-16B) may be configured, by way of example and not limitation, with a single rotor having an output shaft that is parallel to the motor output shaft.

FIG. 1B is a block diagram depicting an exemplary AMFM system. In an exemplary system 101, the controller 125 of the AMFM includes a processor 126. The processor 126 may, for example, include one or more processors. The processor 126 is operably coupled to a memory module 127 and a data store 128. The memory module 127 may, for example, include one or more random-access memory modules. The data store 128 may, for example, include one or more non-volatile memory modules. The data store 128 may, for example, store one or more programs of instructions which, when executed by the processor, cause operations to be performed to control an AMFM 120 (e.g., by controlling current to the first rotor 130 and/or the second rotor 140).

The controller 125 is operably coupled (e.g., electronically and/or mechanically) to the first rotor 130 and the second rotor 140. In response to a command signal (e.g., corresponding to commanded motor output), the controller 125 generates rotor signals (e.g., corresponding to I₁ and I₂) to the first rotor 130 and the second rotor 140. As depicted, the controller 125 may selectively and controllably provide energy (e.g., electric power) from an energy source 160 to the first rotor 130 and/or the second rotor 140. The first rotor 130 and the second rotor 140 electromagnetically interact with an external magnetic field 165 based on the rotor signals and the external magnetic field 165. For example, the external magnetic field 165 may include an MRI magnetic field (e.g., B₀ in FIG. 1A). In some implementations, for example, an MRI magnetic field may be generated using superconducting circuit(s).

The controller 125 is operably coupled, in the depicted example, to a sensor(s) 170. The sensor(s) 170 may, for example, generate feedback signal(s) to the controller 125. The controller 125 may generate the rotor signals based on the command signal and the feedback signal(s) from the sensor(s) 170. In some embodiments, for example, the sensor(s) 170 may generate feedback signal(s) corresponding to a strength and/or direction of the external magnetic field 165. In some embodiments the sensor(s) 170 may generate feedback signal(s), for example, corresponding to an orientation of the first rotor 130 and/or the second rotor 140. In some embodiments, by way of example and not limitation, the sensor(s) 170 may generate feedback signal(s) based on an output of the first rotor 130 and or the second rotor 140. In some embodiments, by way of example and not limitation, the sensor(s) 170 may generate feedback signal(s) based on the orientation of the AMFM with respect to the ambient magnetic field direction.

In the depicted example, the controller 125 is operably coupled to a rotor coil(s) 175 of each of the first rotor 130 and the second rotor 140. For example, the controller 125 may control a magnetic field strength generated by the coil(s) 175. As an illustrative example, the controller 125 may control the magnetic field strength by directly and/or indirectly controlling a current in the coil(s) 175, as disclosed at least with reference to FIG. 1A.

The coil(s) 175 is mechanically coupled (e.g., directly, indirectly) to a power transfer module 180. In some embodiments the power transfer module 180 may include a corresponding output shaft (e.g., as depicted in FIG. 1A).

Each power transfer module 180 is mechanically coupled to a power mixing module 190. The power mixing module 190 may combine mechanical output power from each of the coil(s) 175 to generate a combined output power (e.g., corresponding to a total output moment). As depicted in FIG. 1A, the power mixing module 190 may include, for example, the first gear 135, the second gear 145, and the output shaft 150.

Various embodiments include a motor that uses an ambient magnetic field as a stator field to produce electromagnetic actuation. The motor may include two or more rotors. Each rotor may have its own rotor coils(s) (e.g., armature winding(s)). A shaft axis of one rotor may be nonparallel to a rotor shaft axis of the other rotor. The output of each rotor shaft may be mechanically coupled to produce a common rotary output along a motor output shaft. Such embodiments may advantageously provide an electromagnetic motor that uses an ambient magnetic field for actuation Using two rotors whose shaft axis are nonparallel may advantageously enables a motor to generate torque and rotary motion on the motor output shaft for substantially any orientation of the motor relative to the ambient magnetic field.

FIG. 2A and FIG. 2B depict a perspective view and a top view, respectively, of an exemplary AMFM 200. As shown in FIG. 2A, the AMFM 200 includes a first rotor 205 and a second rotor 210. An ambient magnetic field 215 may provide a stator field used by each of the rotors 205, 210. For example, the ambient magnetic field may be generated by the stationary B₀ field of a magnetic resonance imaging (MRI) scanner (e.g., the MRI scanner 110 as disclosed at least with reference to FIG. 1A).

The rotors 205, 210 each include a rotor armature 220. For example, the rotor armature 220 may include rotor windings (e.g., an electric conducting loop). In the depicted example, electric current is supplied to each rotor armature 220 through motor leads 225. Currents supplied to the motor leads 225 may be transferred to windings in the rotor armature using an electric power transfer device 230 as shown in FIG. 2B. In various embodiments, the electric power transfer device 230 may include a brush/mechanical commutator pair that electrically connects the rotating rotor armature 220 to the motor leads 225. The power transfer device 230 may, for example, provide commutation of the corresponding rotor armature 220.

In some embodiments, the electric power transfer device 230 may include a slip ring assembly that provides a continuous electrical connection between each winding of the rotor armature 220 and each motor lead 225. Commutation in such embodiments may, for example, be achieved using switching circuitry. Such commutation schemes may be referred to herein as “electrical commutation.”

When electrical current is supplied to the rotor winding of the rotor armature 220 in the depicted example shown in FIGS. 2A-2B, the ambient magnetic field 215 and the current in the rotor armature winding may induce a torque on the rotor armature 220. For example, the rotor armature 220 may produce a torque output along a longitudinal axis of respective rotor shafts 235, 240.

As shown, the rotor shaft 235 of rotor 1 and the rotor shaft 240 of rotor 2 are not parallel. A mechanical coupler 245 may, for example, convert mechanical output from each rotor 205, 210 to a common motor output shaft 250. The direction Xₘ of the motor output shaft 250 may, for example, differ from an axis X₁ of the rotor shaft 235 and an axis X₂ of the rotor shaft 240. In various embodiments, the mechanical coupler 245 may include gearing to combine rotational speeds of the rotor shaft 235 and the rotor shaft 240. For example, the speeds of the rotor shaft 235 and the rotor shaft 240, and the output motor shaft 250 may be different.

In various implementations, the AMFM 200 may include an actuator including the output motor shaft 250 extending along the longitudinal axis Xₘ and coupled to be driven by at least one of the rotors 205, 210 configured to rotate about at least one rotation axis intersecting the longitudinal axis. For example, the rotors 205, 210 may be configured to be selectively rotated by the ambient electromagnetic field 215.

Without being bound by a particular theory, various embodiments may operate such that (1) a stationary ambient magnetic field that is external to the motor housing as the stator field; (2) two independent rotors whose rotor shaft axes are nonparallel, and (3) a mechanical coupler to produce a common motor shaft output, can produce rotary torque about the motor shaft output for any orientation of the motor with respect to the ambient magnetic field direction. If a single armature winding on one of the rotors 205, 210 in FIGS. 2A-2B is considered, as an illustrative example, a torque vector (\vec{T}) on that armature loop may be given by \vec{T}=\vec{M}×\vec{B} where \vec{M} is the magnetic dipole moment generated by the winding and \vec{B} is the ambient magnetic field that is used as the stator field (e.g., the ambient magnetic field 215). The dipole moment generated by the winding loop may be \vec{M}=nI\vec{A} where n is the number of turns in the winding, l is the electrical current in the winding, and \vec{A} is a vector whose magnitude is the cross-sectional area of the armature winding and direction is normal to the plane defined by the winding.

The torque component directed along the rotor shaft axis may be what produces the usable rotary torque. The magnitude of the torque about the shaft may be given by Equation 1.

$${T}_{s} = \hat{s} \cdot ( \vec{M} \times \vec{B} ) , \qquad EQUATION 1$$

where ŝ is a unit vector along the direction of the rotor shaft and · denotes the dot product.

In a case where the ambient magnetic field is supplied by an MRI scanner, inside the MRI scanner, the magnetic field term (\vec{B}) in Equation 1 may be dominated by the main stationary field, which is commonly denoted by B₀. If a coordinate system is selected so that the B₀ field is oriented along the z direction, Equation 1 simplifies to Equation 2.

$${T}_{s} = ( {M}_{y} cos ϕ - {M}_{x} sin ϕ ) {B}_{0} sin θ , \qquad EQUATION 2$$

where ϕ and θ are the azimuthal and polar angles specifying the orientation of the rotor axle in spherical coordinates, B₀ is the magnitude of the static magnetic field, and Mₓ and M_{y} are the x and y components of the dipole moment generated by the current in the rotor armature winding.

Equation 2 shows that the shaft torque produced by each rotor may not only depend on the orientation and current in the rotor armature winding (which may determine the magnitude of Mₓ and M_{y} and may be controlled by the motor commutation scheme) but also may depend on the orientation of the rotor shaft with respect to the B₀ field direction. Equation 2 shows that when the rotor shaft is parallel to the stationary B₀ field (i.e., θ=) 0°, the shaft torque generated by current in the armature winding may be zero (Tₛ=0). Accordingly, Equation 2 shows, for example, that a zero torque (no torque) may be generated about a rotor shaft if the rotor shaft axis is parallel to the ambient magnetic field direction.

A motor with two rotors where the shaft axes of each rotor are not parallel (such as depicted in FIGS. 2A-2B) may be configured such that the motor always has one rotor shaft that is not parallel to the ambient magnetic field direction. Such embodiments may ensure that at least one of the two rotors will always be oriented so that a current supplied to its rotor armature can produce a torque about its rotor axis. Thus, the electric current in the armature windings of both rotors may be advantageously controlled to generate a torque on the motor output shaft for various (e.g., any) rotational alignment of the motor with respect to that ambient stator field.

Accordingly, various embodiments may use an ambient magnetic field produced external to the motor as the stator field. Various embodiments may have a motor design including two rotors connected to two separate and non-parallel shafts that are mechanically coupled to produce a common rotary output. The two rotors with non-parallel shaft axes combined with properly commutating each rotor armature may advantageously enable the motor of such embodiments to operate for any arbitrary orientation of the motor with respect to the direction of the ambient magnetic field.

FIG. 3A and FIG. 3B depict an exemplary AMFM including coupling gears for coupling of mechanical output from each rotor. As shown in FIG. 3A, an AMFM 300 includes a first rotor 305 and a second rotor 310 coupled (e.g., rotatably coupled via bushings and/or bearings) to a motor housing 315. Each of the rotors 305, 310 includes an armature winding 320 electrically coupled to corresponding motor leads 325 through an electric power transfer device 330 (as shown in FIG. 3B). The electric power transfer device 330 may, by way of example and not limitation, be a slip ring assembly that advantageously provides independent and continuous electrical connections to each armature winding on the rotor. In some embodiments, commutation of each rotor armature may be achieved using switching circuitry.

In some embodiments, the electric power transfer device 330 may include brushes and a mechanical commutator that is used to supply current to the armature. In some embodiments, the electric power transfer device 330 may be configured to inductively couple the motor leads 325 to the armature windings 320. For example, the inductive coupling may advantageously enable inductively generated currents in the rotor armature. Such embodiments may, for example, advantageously eliminate the need for power leads supplying current to the armature windings 320 in the rotating rotors 305, 310.

In this example, the AMFM 300 further includes a first gear 335 and a second gear 340. The first gear 335 is coupled to a rotor shaft 350 that connects to the output motor shaft 250. The second gear 340 is coupled to a rotor shaft 360. The first gear 335 and second gear 340 are mechanically coupled such that rotation of the first gear 335 results in rotation of the second gear 340. For example, in operations, the gears 335, 340 may advantageously supply output torque to the output motor shaft 250 at any orientation of the motor housing 315 with respect to the ambient magnetic field direction (e.g., the ambient magnetic field 215).

As shown in FIG. 3B, the first rotor 305 includes a rotor shaft 350 supported by shaft supports 355, 375, and the second rotor 310 includes a rotor shaft 360 supported by shaft supports 365, 370. The output motor shaft 250 is also supported by a shaft support 375. The rotor shaft 350 and the rotor shaft 360 are separated by an angle γ. For example, 0°<γ180°. In operation, when the AMFM 300 is placed in an ambient magnetic field, for example, at least one of the rotor shafts 350, 360 may intersect a direction of the ambient field. Accordingly, the AMFM 300 may advantageously supply mechanical power independent of an orientation of the motor housing 315 by arranging at least one axis of rotor shafts to intersect with another axis of at least one of the rotor shafts.

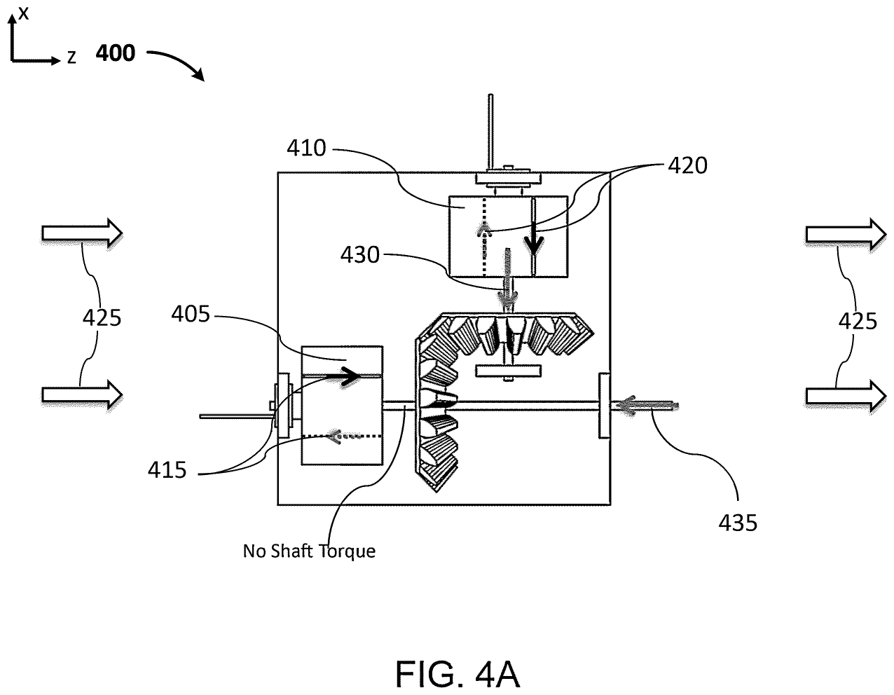

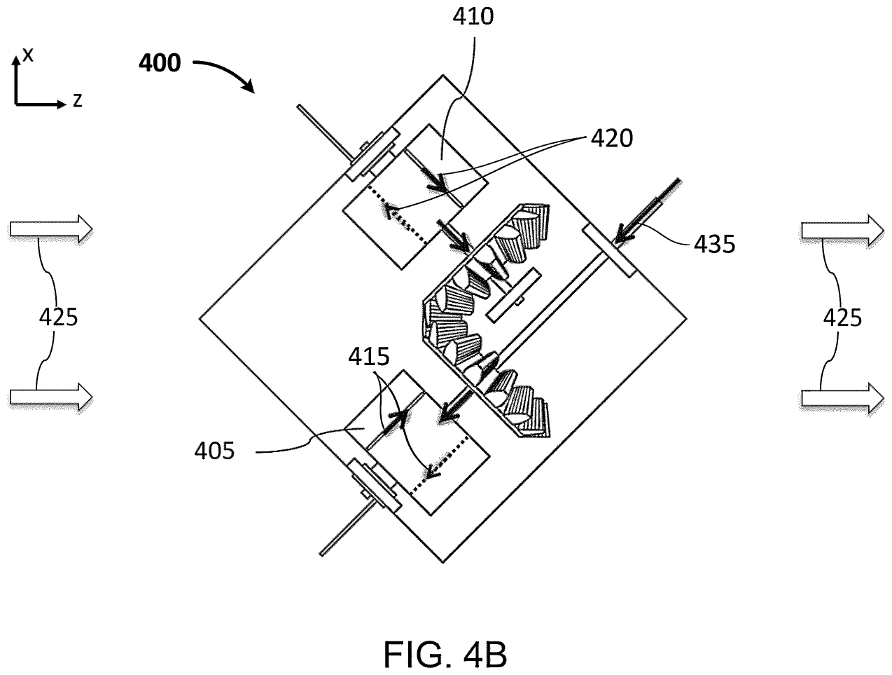

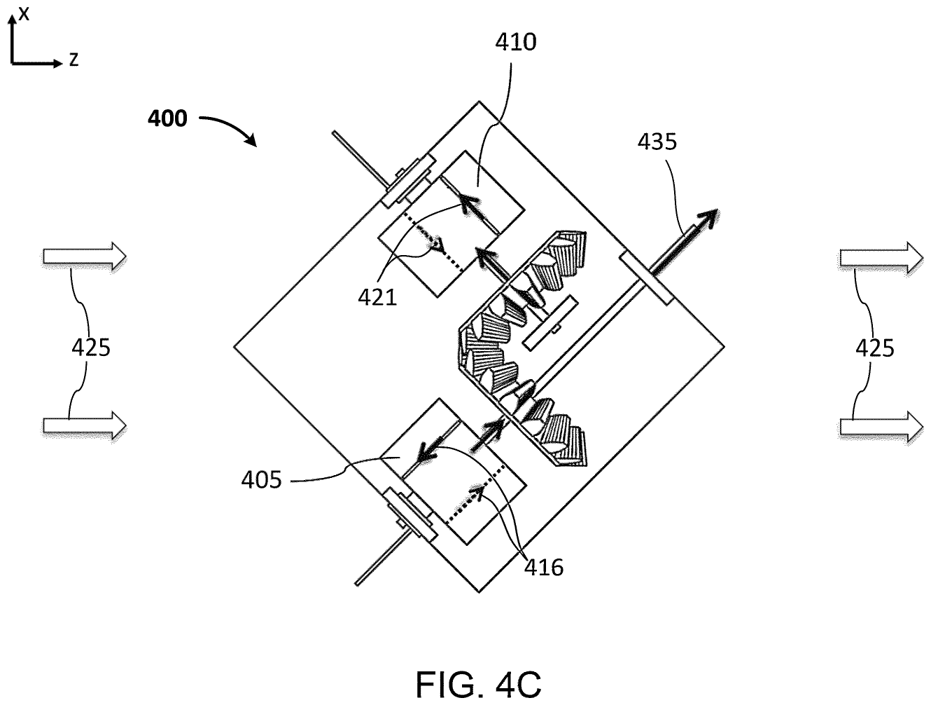

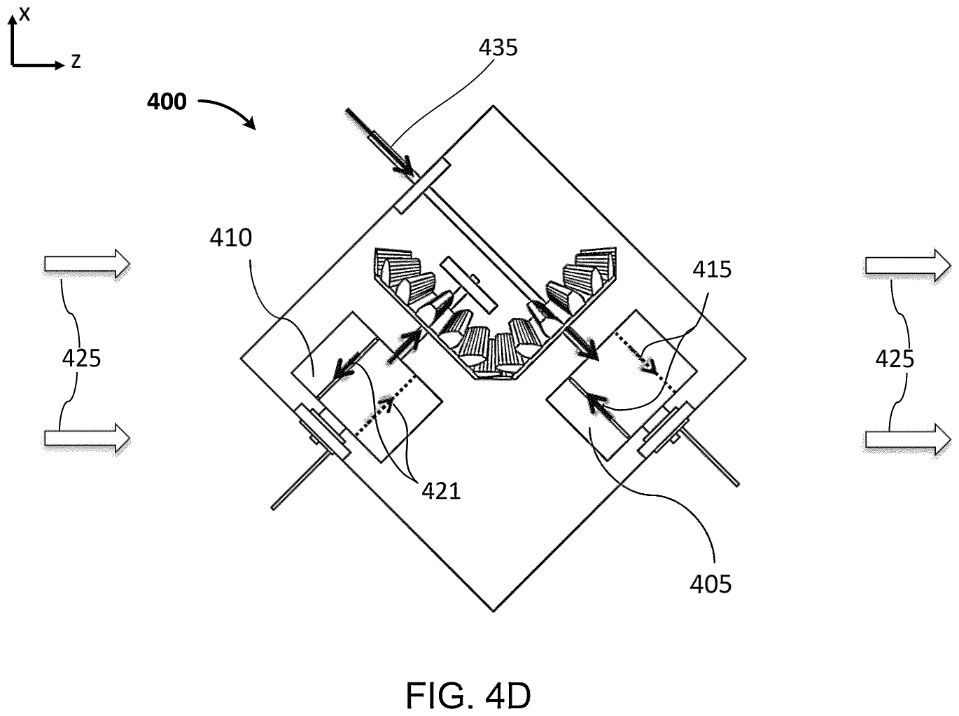

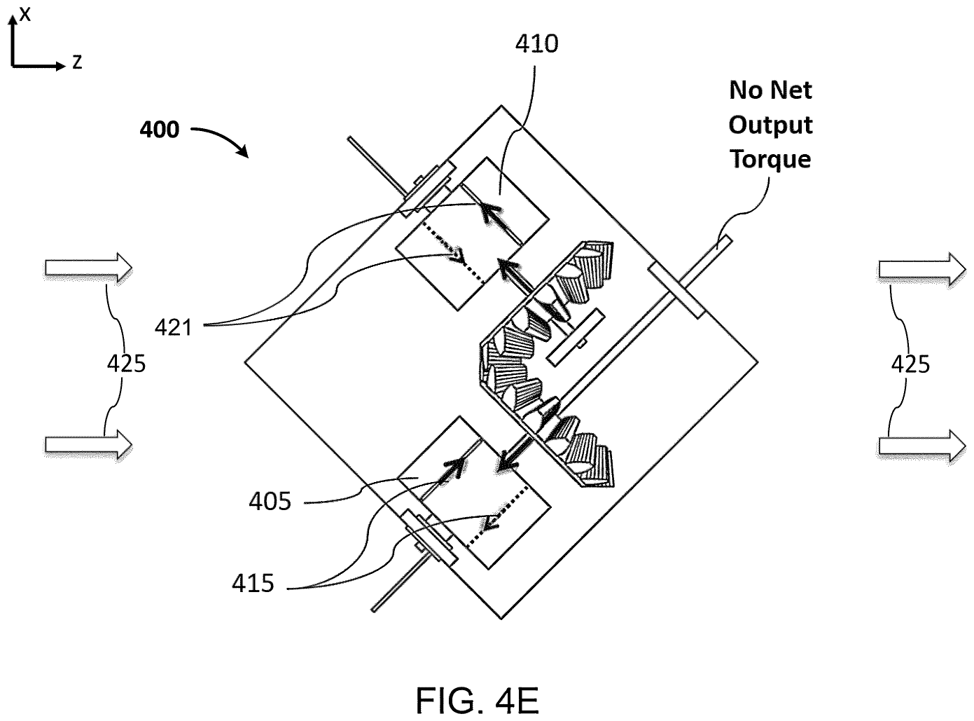

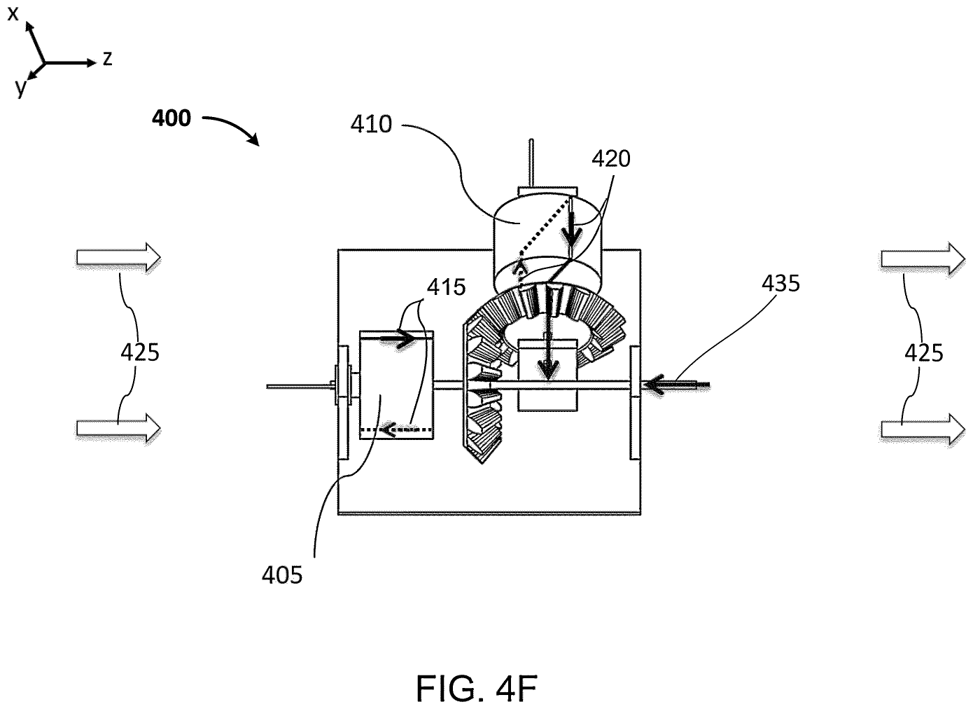

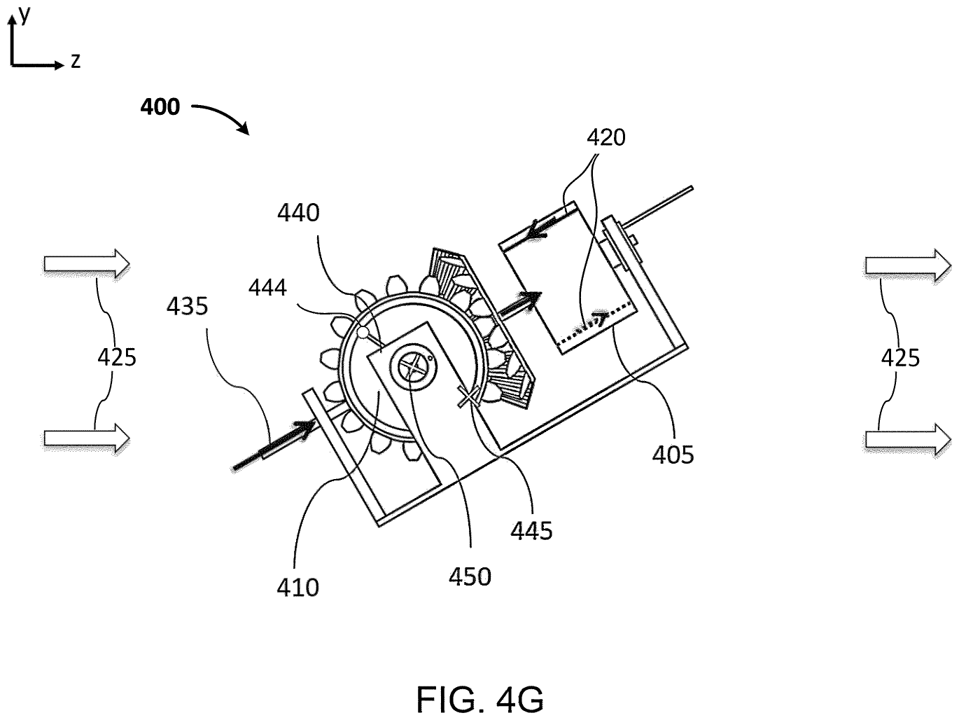

FIG. 4A, FIG. 4B, FIG. 4C, FIG. 4D, FIG. 4E, FIG. 4F, and FIG. 4G depict operations of an exemplary AMFM in exemplary orientations relative to an ambient magnetic field.

In FIGS. 4A-4G, an AMFM 400 includes two rotors 405, 410. In this example, the rotor 405 is supplied with electric current in a current direction 415 (clockwise when viewed from above) or 416 (counterclockwise when viewed from above) in its rotor armature winding. The rotor 410 is supplied with electric current in a current direction 420 (clockwise when viewed from above) or 421 (counterclockwise when viewed from above) in its rotor armature winding. The AMFM 400 is placed in various orientations with respect to an ambient magnetic field 425. The ambient magnetic field 425 may, for example, be in a direction to induce torque 430 at rotor shafts of the rotors 405, 410 depending on the orientation. A net output torque 435 is produced at a motor shaft of the AMFM 400.

In FIGS. 4A-4G, each rotor armature is depicted as a single loop for conceptual simplicity. In various embodiments (not shown) each rotor armature may include, by way of example and not limitation, 3 distinct armature windings separated by 120°.

FIG. 4A shows a case where the rotor 405 shaft axis is parallel to the ambient magnetic field 425. As depicted, the electric current in current direction 415 may produce no output torque about the rotor 405 shaft axis. However, the rotor 410 shaft axis is not parallel to the ambient magnetic field 425. For example, the rotor 410 may produce a torque 430 about the rotor 410 shaft axis for the electric current direction 420. As shown, a net output torque 435 is generated.

FIGS. 4A-4E depict different orientations of the motor where it has been rotated about the y-axis or different current directions in the rotor armature. For the current directions 415, 420 shown in FIG. 4B, both rotors 405, 410 contribute to the net output torque 435. FIG. 4C shows that reversing the current direction in both rotors reverses the direction of the motor output torque. The reversed current directions 416, 421 are shown in FIG. 4C. FIG. 4D depicts a different motor orientation. For the current directions 415, 421, both rotors contribute to the net output torque 435. FIG. 4E shows that the current directions 415, 421 in rotor 1 and rotor 2 may be controlled (e.g., purposely, accidentally) via a commutation scheme such that each rotor produces an output torque that will be counteracted by the other rotor. Exemplary commutation schemes are described with reference to FIGS. 8A-10B. In this instance, no net output torque is generated on the motor shaft. FIG. 4F depicts a motor orientation where it has been rotated about the z-axis with respect to the configuration in FIG. 4A. FIG. 4G depicts a motor orientation where it has been rotated about the x-axis with respect to the configuration in FIG. 4A. As shown in FIG. 4G, the rotor 410 is supported by a frame 440. The rotor 410 includes an electric current 444 flowing in a direction oriented out of the page, and an electric current 445 flowing into the page. The rotor 405 includes an electric current direction 420. In this example, a resulting torque 450 from the combined output of rotor 410 and rotor 405 is directed into the page. All the exemplary cases in FIGS. 4A-4G demonstrate that the AMFM 400 may operate in any orientation rotated about the x-axis, y-axis, and z-axis with respect to the ambient field.

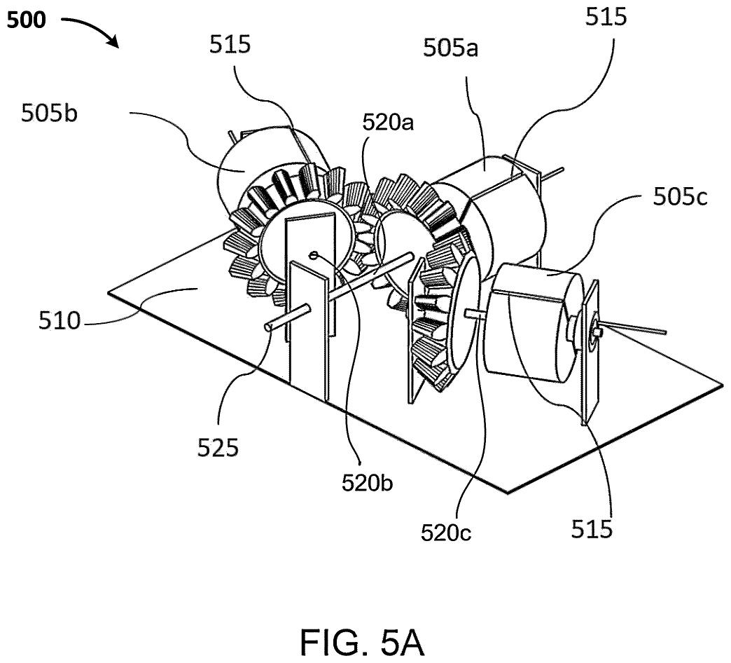

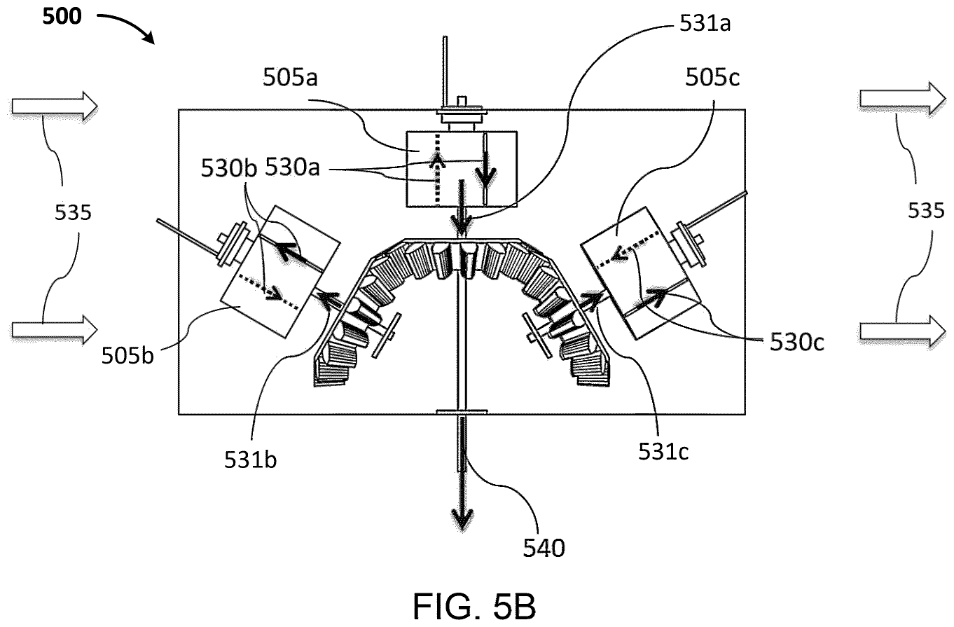

FIG. 5A and FIG. 5B depict an exemplary 3-rotor AMFM 500. The 3-rotor AMFM 500 includes three rotor units 505a, 505b, 505c coupled to a motor support 510. Each of the rotor units 505a, 505b, 505c includes an armature 515 and a rotor shaft 520 (e.g., 520a, 520b, 520c). In this example, orientation angles of each rotor shaft 520 may be different.

The 3-rotor AMFM 500 includes a motor output shaft 525. In various such embodiments, a torque constant (k_{T}) at the motor output shaft 525 may be maintained within a predetermined range when an orientation of the 3-rotor AMFM 500 changes with respect to the ambient magnetic field.

FIG. 5B shows a top view of the 3-rotor AMFM 500. In this example, the 3-rotor AMFM 500 receives current (shown with arrows labeled with directions 530a, 530b, 530c) in all three rotors 505a-c. Based on the current directions 530a, 530b, 530c and a direction of an ambient magnetic field 535, the rotors 505a-c, for example, may be controlled to contribute to supply a common output torque 540 along the motor output shaft 525. For example, rotor unit 505a may contribute an output torque 531a. Rotor unit 505b, for example, may contribute an output torque 531b. Rotor unit 505c, for example, may contribute an output torque 531c. Power transmitters (e.g., gears) coupled to the respective output shafts 520a, 520b, and 520c combine the individual output torques 531a, 531b, and 531c to generate the common output torque 540. Various applications may benefit from a less varying output generated using 3 or more rotors.

FIG. 6 depicts an exemplary result 600 of a theoretical torque constant (k_{T}) for a 1-rotor motor, a 2-rotor motor, and a 3-rotor motor. For example, the 2-rotor torque constant may be generated by the AMFM 300 as described in FIGS. 3A-3B, where γ=90°. For example, the 3-rotor motor result may be generated by the 3-rotor AMFM 500 as described in FIGS. 5A-5B, where the rotor shafts 520 (e.g., 520a, 520b, 520c) are separated by 60°. In some examples, when a motor orientation is varied about the y-axis with respect to an ambient magnetic field (e.g., the ambient magnetic field 535). An angle α is the angle between the ambient magnetic field and the axis of the motor output shaft. For the 1-rotor motor design, the torque constant varies significantly and is zero for certain orientations. For the 2-rotor motor and 3-rotor motor designs, the torque constant variability is advantageously smaller. As shown in FIG. 6, the torque constant is always nonzero for the 2-rotor motor and 3-rotor motor designs.

In various embodiments a motor control (e.g., as disclosed at least with reference to FIGS. 2A-5B) may include an electric power transfer device in which each rotor uses brushes and a mechanical commutator. In such embodiments, power may be supplied to the rotor armature using brushes and a mechanical commutator.

In various embodiments a motor control (e.g., as disclosed at least with reference to FIGS. 2A-5B) may include an electric power transfer device in which each rotor uses slip rings. In such embodiments, power may, for example, be supplied to the rotor armature using slip rings. Commutation may, for example, performed electrically using switching circuitry.

FIG. 7A depicts an exemplary rotor unit 700 having a brush and mechanical commutator 705. As depicted, the commutator 705 is divided in two conducting arcs 706a, 706b that are separated by an insulating element 710 (e.g., an insulator, an air gap). In some implementations, the commutator 705 rotates with a rotor 715. As shown in FIG. 7A, the exemplary brush and mechanical commutator 705 includes two conducting arcs contacting the brushes 725, 730. Each brush 725, 730 is connected to a separate electrical lead 735, 740. In this example, a rotor armature 745 is electrically connected to conducting segments of the commutator 705 (e.g., so that electric current can flow from the electrical leads 735, 740 to windings on the rotor armature 745).

FIG. 7B shows an exemplary mechanical commutation scheme for a rotor 701 having three rotating coils that are separated by 120°. In this example, a commutator 750 is divided into 3 conducting segments 755a, 755b, 755c separated by the insulation elements 710. Three rotor loops 760 (e.g., 760ab, 760bc, 760ca) are electrically connected to the conducting segments 755a, 755b, 755c as shown. Each of the conducting segments 755a, 755b, 755c may contact one of two brushes 725, 730 to electrically connect a rotor loop to the electrical lead 735, 740. Each of the brushes 725, 730 may be configured with respect to a static ambient magnetic field 765 such that the brushes 725, 730 are in line with the ambient magnetic field direction. In various embodiments, electrical currents to each rotor are provided by leads 735, 740. Proper commutation is, in the depicted example, achieved using the included brushes 725, 730, orientation of mechanical commutator 750 and the rotor loops 760, and the ambient magnetic field 765.

FIG. 8A shows an exemplary two-rotor AMFM 800 with two Hall effect sensors 805, 810 mounted on a motor housing 815. In this example, two rotors 820, 825 may be configured as disclosed at least with reference to FIGS. 3A-3B, where γ=90° (rotor shaft axes of the two rotors 820, 825 are orthogonal to each other). In the depicted example, the Hall effect sensors 805, 810 are also rotated by this same 90° relative to each other.

In some implementations, to control a net torque output of the two-rotor AMFM 800, current directions of the rotor 820 and the rotor 825 may be determined. For example, in at least some embodiments, because an angle between the rotors 820, 825 is fixed (e.g., γ=) 90°, the current directions may be determined based on an orientation of the motor output shaft in the x-z plane. For example, information about the motor orientation (e.g., a direction vector) with respect to the ambient magnetic field direction (as labeled as a in FIG. 8A) may be dynamically detected using the Hall effect sensors 805, 810 to control currents in the rotors 820, 825.

FIG. 8B shows an exemplary circuit diagram for switching current based on readings from the Hall effect sensors 805, 810 in the AMFM 800 as described with reference to FIG. 8A. In some embodiments, a circuit 830 may be used to switch current to the rotor 820 and the rotor 825 based on readings from the Hall effect sensors 805, 810. The circuit 830 includes 8 switches labeled S1-S8. R1 and R2 denote the rotor 820 and the rotor 825, respectively. Readings from the two Hall effect sensors 805, 810 may, for example, be inputs to a logic circuit (e.g., of the controller 125) which may be configured to control the switching scheme.

In various embodiments, the logic circuit and/or switching may be implemented internal to the motor housing 815. For example, in various such embodiments, the only outside electrical connections to the AMFM 800 may be Terminal A and Terminal B. Terminal A and Terminal B may be connected to a direct current (DC) power supply in some embodiments. In some embodiments. Terminal A and Terminal B may be connected to an H-bridge motor controller, for example. Such embodiments may advantageously allow the motor to be controlled using a traditional brushed DC motor control scheme.

In various embodiments an encoder(s) may be provided on the motor output shaft. The encoder output may, for example, be supplied to an external servomotor controller. Such embodiments may advantageously enable closed loop control.

FIG. 8C depicts an exemplary logic table 840 with illustrative switching logic for the exemplary AMFM 800 as described in FIGS. 8A-8B. The logic table 840, combined with the circuit 830 in FIG. 8B may control currents supplied to armature windings of the rotor 820 and the rotor 825 for any orientation angle α. In the table 840, “+” denotes a high voltage signal, and “o” denotes a low voltage signal from the Hall effect sensors (H1, H2). For the switches “o” means the switch is open and “x” means the switch is closed.

In various embodiments, various control strategies may be implemented. For example, a separate H-bridge controller may be used to provide independent control to each rotor. Measurement of information about the angle α may, for example, then be used to properly control the switching of the two H-bridge motor controllers to achieve a desired control. Position information may, for example, be obtained from a magnetometer and/or using an absolute angular positional encoder sensor.

In various embodiments, motor control may be performed using an electric power transfer device to each rotor using a slip ring assembly. Various such embodiments may advantageously be deployed in use-cases in which the motor needs to operate in any orientation with respect to the ambient magnetic field.

FIG. 9A depicts a schematic of an exemplary 8-conductor slip ring assembly 900. For example, the slip ring assembly 900 is a brushed type. As shown, the 8-conductor slip ring AMFM assembly 900 includes conducting rings 905 attached to a rotating shaft 910. Each of the rings 905 is connected to a corresponding electrical lead 915 that moves with the rotating shaft 910. Each of the conducting rings 905 is paired with a corresponding stationary electric lead 920 and a corresponding brush contact 925. The brush contacts 925 may advantageously enable the stationary electric lead 920 and corresponding electric lead 915 on the rotating shaft 910 to maintain a continuous electrical connection for any rotation of a motor shaft about its axis.

Various embodiments may implement brushless slip rings. Such embodiments may, for example, advantageously reduce electromagnetic noise generated by the brush and conducting ring contact. In an illustrative embodiment of a brushless design, a pool of liquid metal that is molecularly bonded to the contact may be configured to generate a continuous electrical connection.

FIG. 9B depicts an exemplary rotor, rotor axle, and power transfer device for the 8-conductor AMFM system 1005 as described with reference to FIG. 9A and FIG. 9C. The exemplary rotor 935 includes a rotor 935 coupled to the rotating shaft 910. The rotor 935 includes three armature windings 940a, 940b, 940c. The armature windings 940a, 940b, 940c, in this example, are coupled to corresponding stationary leads A, B, C, respectively. As shown, the rotor armature windings 940 (e.g., 940a, 940b, 940c) are configured in a delta configuration on a rotor support structure 945. Various embodiments may implement a wye configuration. In the depicted example, the rotor 935 may include three Hall-effect sensors 950a, 950b, 950c configured to detect an orientation of the rotor with respect to an ambient magnetic field direction. These Hall-effect sensors 950a, 950b, 950c may, for example, be bipolar latching Hall-effect sensors. The Hall-effect sensors 950a, 950b, 950c, as depicted, are powered via a voltage source connected to the V and G (e.g., ground) electrical leads on the slip ring assembly 900. The wire leads H1, H2, and H3 on the slip ring assembly, for example, may transmit signals from the Hall-effect sensors 950a, 950b, 950c to a motor controller (e.g., the controller 125). In various embodiments, a rotor support and/or other materials may be made from non-magnetic and non-ferromagnetic materials.

Some embodiments may, for example, include more than 8 conductors. Some embodiments may, for example, include less than 8 conductors. For example, some embodiments may have a number of conductors related to a number of rotor armature windings.

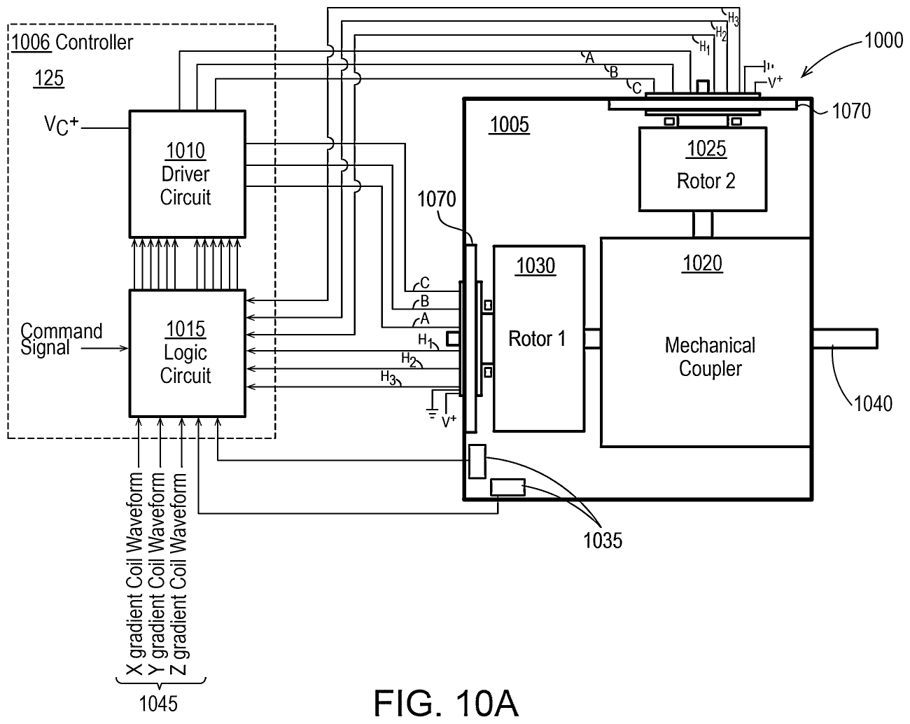

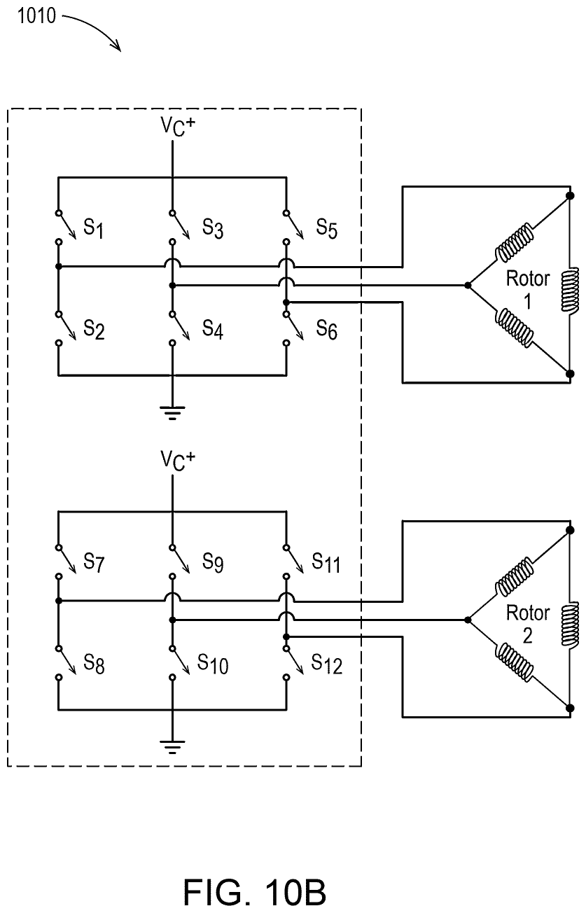

FIG. 10A depicts a block diagram of an exemplary AMFM system 1000. In this example, the exemplary AMFM system 1000 includes a motor housing 1005. The rotor system 1000 is operably coupled to a controller 1006. The controller 125 includes a driver circuit 1010 and a logic circuit 1015. As is shown the rotor housing 1005 includes (e.g., encloses) a mechanical coupler 1020 coupled to a rotor 1025 and a rotor 1030.

In this example, signals generated by the Hall-effect sensors 950a, 950b, 950c on rotor 1 and rotor 2 (6 in total for the depicted configuration) are supplied to the logic circuit 1015 on the controller 1006. Upon receiving a command signal and rotor positional information (determined from the Hall-effect sensors), the logic circuit 1015 may generate control signals to the driver circuit 1010. The driver circuit 1010, for example, may supply a controlled currents to the motor leads (e.g., the motor leads A, B, C shown in FIG. 9B) on the rotor 1025 and the three motor leads (A, B, C) on the rotor 1030.

Various embodiments may implement other methods to determine the orientation of each rotor axis and an orientation of each rotor winding with respect to the ambient magnetic field. Such embodiments may enable the motor controller to provide suitable commutation of each rotor armature.

In this example, the rotor housing 1005 includes two additional Hall-effect sensors 1035. The two additional Hall-effect sensors 1035 may, for example, provide specific information about the orientation of each rotor axle with respect to the ambient magnetic field.

In various embodiments, magnetometers may also be used, for example, to measure a direction and strength of an ambient magnetic field. In some embodiments, a rotary encoder (e.g., an absolute encoder, incremental encoder) on a motor output shaft 1040 may be used, for example, to provide additional information to and/or instead of (at least some of) the Hall Effect sensors. Various embodiments may be configured to measure a back electro-motive-force in unpowered windings. Such embodiment may advantageously provide information about the rotor position to the motor controller. Some embodiments may use field-orientated control and/or other control and/or rotor position sensing hardware and/or algorithms used by brushless direct current (BLDC) and/or brushless alternating current (BLAC) motor controllers.

An exemplary application for AMFMs disclosed at least with reference to FIGS. 2A-10B may include controlling of precise robotic systems inside an MRI scanner during imaging. In operation, for example, an MRI may power strong electromagnets. The electromagnets may, for example, switch at a rate substantially between 100-5000 Hz. Such switching may, for example, induce current in the motor armature and/or motor leads. This induced current may lead to jitter in motion control.

In the depicted example, the controller 125 may receive feed-forward control inputs 1045. As shown, the logic circuit 1015 may receive gradient coil waveforms in (X,Y,Z) directions of an MRI scanner (e.g., the MRI scanner 110). For example, the logic circuit 1015 may use the received gradient coil waveforms information to determine control strategies.

Some embodiments may include a single rotor motor with a torque constant (substantially) unaffected for any in-plane rotation of an output shaft of the motor. Various such embodiments may, for example, still be operated for different orientations of the motor shaft. Such embodiments may, for example, advantageously be implemented to control a robotic stage in which a majority of the motion the motor will experience is in a single plane.

FIG. 10B shows an exemplary driver circuit 1010 of the exemplary AMFM system as described with reference to FIG. 10A. In the depicted example, rotor current is controlled in each rotor armature using a set of 6 switches for a total of 12 switches (labeled S1-S12), which may advantageously be used to control an AMFM system shown in FIG. 10A.

FIG. 11A and FIG. 11B depict an exemplary embodiment with a single rotor motor 1100. As depicted, the single rotor motor 1100 includes a motor housing 1105. The single rotor motor 1100 includes (e.g., in the motor housing 1105) a rotor 1125 connected to a rotor shaft 1115. As shown, the rotor shaft 1115 is not parallel to a motor output shaft 1120. In the depicted example, an axis of rotation of the rotor shaft 1115 intersects an axis of rotation of the motor output shaft 1120. In the depicted example, bearings 1155 are used to support the rotating shafts against the motor housing.

In the depicted example, a rotor armature includes multiple coil loops 1130. In this exemplary embodiment, three bipolar latching Hall-effect sensors 1136 (as shown in FIG. 11B) are placed on the single rotor motor 1100. The three bipolar latching Hall-effect sensors 1136 may, for example, allow a motor controller (e.g., the controller 125) to provide correct electrical commutation (via armature leads 1140) to achieve a selected motor output.

A slip ring assembly 1145 is implemented in the depicted embodiment to electrically connect the coil loops 1130 of the rotor armature and the Hall-effect sensor leads 1135 to the motor controller.

FIG. 11B shows the single rotor motor 1100 with respect to an ambient magnetic field 1150. As shown, rotations of the motor housing 1105 about the y-axis do not change the motor operation or the motor torque constant. For example, in this exemplary 1-rotor design, rotations about other axes may alter the motor torque constant and/or operation characteristics. In various implementations, the ability to operate at many orientations with respect to the ambient magnetic field 1150 may be sufficient. For example, the single-rotor design may advantageously provide reduced complexity. Such embodiments may, for example, decrease cost (e.g., design, manufacturing, maintenance) and/or increase reliability.

As depicted in FIGS. 11A-11B, the motor 1100 includes two miter gears 1110, 1111, to mechanically couple the rotor shaft 1115 to the motor output shaft 1120. In various embodiments, a worm gear coupling, a screw gear, bevel gears, and/or other gearing systems may be implemented.

Various embodiments may be deployed in a non-static ambient magnetic field. The dominating magnetic field of many conventional 1.5 Tesla and 3 Tesla MRI scanners may be static. The dominating magnetic field may, for example, be produced by a superconducting magnet. In various implementations, gradient coils of an MRI scanner can contribute to the ambient magnetic field. In some applications, an ambient magnetic field direction and/or strength may change. Various embodiments disclosed herein (e.g., as disclosed at least with reference to FIGS. 1A-11B) may advantageously be configured for use in circumstances where the ambient magnetic field direction is changing.

FIG. 12 depicts a schematic diagram of an exemplary AMFM closed loop control system 1200. The loop control system 1200 includes a motor controller 1205, an AMFM 1210, and an MRI compatible position encoder 1215. The position encoder 1215 may provide an absolute position and/or incremental rotation of the motor output shaft 1220. The motor controller 1205, for example, may receive command signals externally. Based on the command signals, for example, the motor controller 1205 may generate a control signal to the AMFM 1210 to generate a torque requested by the control signal. Accordingly, for example, the motor controller 1205 may advantageously provide closed loop positioning of the angular rotation of the motor output shaft 1220.

In this example, the MRI compatible encoder 1215 may provide position feedback to the motor controller 1205. Such embodiments may advantageously enable closed-loop control of the motor speed and/or position. Closed-loop control may advantageously enable precisely actuating a robotic system. The MRI compatible encoder 1215 may detect the position information of the output shaft 1220 using, for example, optical and/or magnetic methods. In various implementations, the motor controller 1205 may advantageously use the position feedback information to advantageously mitigate electromagnetic noise that degrades MRI image quality.

In various implementations, MRI systems may be extremely sensitive to radio-frequency noise. For example, electromagnetic noise generated near the Larmor frequency may significantly degrade MRI image quality. Various embodiments (e.g., configured such as disclosed at least with reference to FIGS. 2A-12) may be configured as MRI-compatible motors with reduced electromagnetic energy radiated by the motor to advantageously reduce electromagnetic noise that degrades MRI image quality. Such embodiments may advantageously enable simultaneous actuator operation and imaging. FIG. 13 depicts an exemplary MRI compatible AMFM control system (MCACS). In this example, a MCACS 1300 includes a motor controller 1305, switching circuitry 1310, power supply inverters 1315, and associated hardware (not shown) enclosed in a conducting enclosure 1320. For example, the conducting enclosure 1320 may advantageously minimize radiated electromagnetic noise.

As shown, the MCACS 1300 is coupled to an AMFM 1325 using cables 1330. In various embodiments, the cables 1330 connecting the motor to the motor controller may be shielded with a conducting shield 1340 that is electrically connected to the conducting enclosure 1320. In the depicted example, radio frequency traps 1335 along a length of the cables 1330 are placed. These traps may, for example, be configured to suppress common mode currents on a cable shield 1340 that radiate at the Larmor frequency. In the depicted example, slip rings 1336 are used to electrically connect wires in cabling 1330 to rotor.

As depicted in FIG. 13, the AMFM 1325 is housed in a conducting enclosure 1345. As is depicted in FIG. 13, the conducting enclosure 1345 is electrically connected to the cable shield 1340. The conducting enclosure 1345 may advantageously prevent radiated electromagnetic noise (e.g., from brushes) to reduce radiated electromagnetic noise that may be produced from the current flowing at each rotor.

The AMFM 1325, in this example, includes a motor shaft 1350 that penetrates the conducting enclosure 1345. In some implementations, the motor shaft 1350 may be constructed from a low conductivity material (e.g., fiberglass, carbon fiber, titanium). Such embodiments may, for example, advantageously prevent the shaft from acting as an antenna to radiate noise from inside the conducting enclosure 1345 around the motor to an MRI scanner 1355. In FIG. 13 the B₀ field of the MRI is shown by arrow 1360.

Various embodiments may include a miniaturized motor. A miniaturized motor may, for example, advantageously enhance an electromagnetic interference reduction strategy. A reduced size (e.g., miniaturized) motor (e.g., an AMFM) may reduce a size of the conducting enclosure 1345 used to shield the motor. Reduction of conducting material may advantageously reduce or eliminate degradation of image quality, such as from eddy currents. In combination with a non-conducting or low-conductivity motor shaft penetrating the motor housing and actuating the robotic system, radiation of electromagnetic noise from the housing may advantageously be reduced or prevented.

FIG. 14A, FIG. 14B, and FIG. 14C depict an exemplary implementation of an AMFM in the patient area of an MRI scanner. In this example, an MRI scanner 1400 includes a scanner bore 1405. For example, a patient 105 within the scanner bore 1405 may be situated during imaging. The MRI scanner 1400, for example, may produce a strong static magnetic field 1410 (or B₀ field). The strong static magnetic field 1410 may, for example, be oriented along the length of the MRI scanner bore 1405. The strong static magnetic field 1410 (whose direction is shown by arrows in FIGS. 14A-14C) provides a stator field for an AMFM 1415 in the scanner bore 1405. For example, the AMFM 1415 may be used to actuate the robotic system 1420.

As shown in FIG. 14B, the robotic system 1420 may include an end effector 1425. The end effector contains a surgical instrument guide 1485 which may be used, for example, to precisely place surgical instruments and/or needles, and/or to perform drilling during image-guided surgical interventions. In this example, the end effector 1425 has 2 independent degrees-of-freedom (first degree-of-freedom (first DOF 1435), and second DOF 1430). Exemplary AMFM disclosed at least with reference to FIGS. 1A-13 may, for example, be implemented for controlling at least the second DOF of the end-effector.

To control the first DOF, an AMFM 1440 may, for example, be a single rotor actuator where a rotor shaft and a motor output shaft directions are the same. For example, the static B₀ field 1410 of the MRI scanner 1400 may provide a stator field that is used for actuation. In some implementations, the AMFM 1440 may be enclosed within a housing made of a conducting material to advantageously minimize electromagnetic interference (EMI) between the AMFM 1440 and imaging systems in the MRI scanner 1400.

As shown, a rotation of the first DOF 1435 of the end effector 1425 may cause an orientation of a motor output shaft of an AMFM 1450 used to control the second DOF 1430 to change with respect to the ambient magnetic field 1410. Actuation of the second DOF 1430 may be provided regardless of the position of the first stage by various AMFM embodiments disclosed at least with reference to FIGS. 1A-13 in this disclosure.

In the depicted example, an AMFM 1450 is disclosed at least with reference to FIGS. 11A-11B is implemented to actuate the final stage of the end effector 1425. The AMFM 1450 includes a single rotor 1460 and rotor axle 1445 in which a shaft axis of the rotor 1460 is perpendicular to a motor output shaft 1465. Gearing 1455 may be provided, for example, to transform an output direction. In the depicted example, a slip ring assembly 1470 is used to transfer power to the rotor windings and electrical commutation is achieved using switching circuitry. Although the AMFM disclosed at least with reference to FIGS. 11A-11B is depicted, other AMFM embodiments may be used (e.g., the AMFM embodiments disclosed at least with reference to FIGS. 2A-2B).

As shown in FIG. 14C, an exemplary robotic system 1475 may be used to move to different surgical targets. Linear translation of the robotic system 1475 along the x and z axis, as well using the AMFM 1450 of the end effector 1425 may be used to move from a surgical instrument guide trajectory to a different surgical instrument guide trajectory. This functionality may, for example, advantageously help enable robotically assisted image-guided surgical procedures. Although the exemplary depicted robotic system is shown in the context of a neurosurgery application, various embodiments may be implemented for various surgical and/or anatomical targets (e.g., that may benefit from the use of real-time magnetic resonance imaging during surgery).

FIG. 15A and FIG. 15B depict an exemplary hybrid AMFM (h-AMFM). In this example, a h-AMFM 1500 includes conducting rotor windings 1505 wound on a non-magnetic support structure 1510. In this example, a mechanical commutator 1515 and brushes 1540 are used to supply current to the rotor windings 1505. In some implementations, slip rings and electrical commutation may be used. Mechanical output produces rotary motion along a rotor axle 1520.

The h-AMFM 1500 also includes an auxiliary stator winding 1525 wound on a non-magnetic support structure (an auxiliary stator 1530) that is fixed in position relative to a motor housing 1535, brushes 1540, and/or an axis of the rotor axle 1520. For example, the auxiliary stator 1530 may be used as the auxiliary stator 158 described with reference to FIG. 1A. The auxiliary stator 1530 may, for example, produce a magnetic field (an auxiliary stator field 1545) predominantly along a direction shown by arrows. In some implementations, the magnetic field may be produced when terminals Sa and Sb are connected to a voltage difference.

In some implementations, the h-AMFM 1500 may be included in a bore of an MRI scanner. As shown, an ambient field 1550 may be used to induce motion of the rotor when the h-AMFM 1500 is near a superconducting magnet 1555 of the MRI scanner. For example, the auxiliary stator windings 1525 may optionally be powered in this use case.

In some implementations, the h-AMFM 1500 may advantageously leverage a performance benefit (e.g., small footprint high toque) enabled by using the strong magnetic field 1550 of the MRI scanner for actuation. For example, at the same time, the h-AMFM 1500 may still provide a flexibility to operate when the h-AMFM 1500 is positioned in locations where the ambient magnetic field 1550 produced by the MRI is weak. In some implementations, the h-AMFM 1500, having the dual auxiliary stators 1530, may be actuated in weak ambient field 1550 because the stator field provided by the separate auxiliary stator winding 1525 may be oriented such that the interaction between its contribution to the stator field may be optimized for certain orientation angles of the actuator (i.e., to have the motor axle 1520 parallel to ambient magnetic field).

In various implementations, a h-AMFM may include at least one separate auxiliary stator winding. The orientation of the auxiliary stator winding may, for example, be fixed relative to the axis of rotation of one rotor in the AMFM. For example, an orientation of the auxiliary motor stator winding relative to the rotor axis may be determined based on an orientation of the motor housing relative to the ambient magnetic field. In some implementations, magnetic field sensors such as Hall Effect sensors may be mounted to the motor housing and/or rotor to measure the orientation of the ambient magnetic field with respect to the auxiliary stator windings and the rotor windings, respectively. For example, the current to the auxiliary stator windings and/or rotor may be selectively controlled based on the orientation and strength of the ambient magnetic field and rotor position to achieve optimal control for any positioning and orientation of the system.

As an illustrative example, FIG. 15B shows a scenario where the h-AMFM 1500 is located far from the ambient magnetic field 1550 of the MRI scanner. For example, the stator field produced by the MRI scanner may be negligibly small and insufficient to produce the required torque output. In this scenario, the auxiliary stator field 1545 may be generated in a sufficient magnitude to interact with currents in the rotor winding to induce a torque on the rotor and to rotate the motor axle 1520.

In various implementations, the h-AMFM 1500 may be required to actuate robot systems located in regions where the MRI's ambient magnetic field is weak. For example, in an emergency situation where a robot system needs to be actuated far from an ambient magnetic field of an MRI system. For example, the robot systems may be used outside of the MRI environment for procedures or testing. In some examples, an AMFM may be required to produce a desired torque in different orientations with respect to the MRI scanner. When, for example, the AMFM may be on or near the end effector of a robotic system, the AMFM may be required to operate irrespective of its orientation in the magnetic field.

FIG. 16A and FIG. 16B depict an exemplary h-AMFM 1600 with a coreless rotor 1605. As shown in FIG. 16A, the h-AMFM 1600 includes, as shown, auxiliary stator coils 1610 inside the coreless rotor 1605. As shown, the auxiliary stator coils 1610 are completely enclosed within the coreless rotor 1605. In operation, similar to the h-AMFM 1500 described in FIGS. 15A-15b, the h-AMFM 1600 may use a combined stator field produced by an ambient magnetic field 1550 (i.e. MRI scanner) and the field 1615 produced by the auxiliary stator coils 1610 wound around an auxiliary stator support 1620. For example, the auxiliary stator support 1620 may be non-magnetic. In some examples, the h-AMFM 1600 may be more compact and efficient in space usage.

In the depicted example, the coreless rotor 1605 is configured to rotate about the auxiliary stator support 1620 and the auxiliary stator coils 1610. The rotation is about axle 1520. The axle 1520 is connected to the coreless rotor 1605. The axle is rotatably coupled via bushings and/or bearings 1621 to the auxiliary stator support 1620 to allow the axle 1520 and coreless rotor 1605 to rotate with respect to the auxiliary stator.

As shown in FIG. 16B, a h-AMFM 1630 includes secondary stator winding 1635 with a winding pattern in both interior to an exterior to the coreless rotor 1605. The addition of the windings external to the coreless rotor 1605 may, for example, act in lieu of a ferromagnetic motor housing to advantageously control and focus direction of the field 1615. Accordingly, the addition of winding external to the coreless rotor 1605 may advantageously improve motor performance by enhancing the field 1615. For example, the h-AMFM 1630 may be provided, in some implementations, with a non-ferromagnetic housing 1640 (e.g., having negligible magnetic susceptibility in a strong ambient magnetic field).

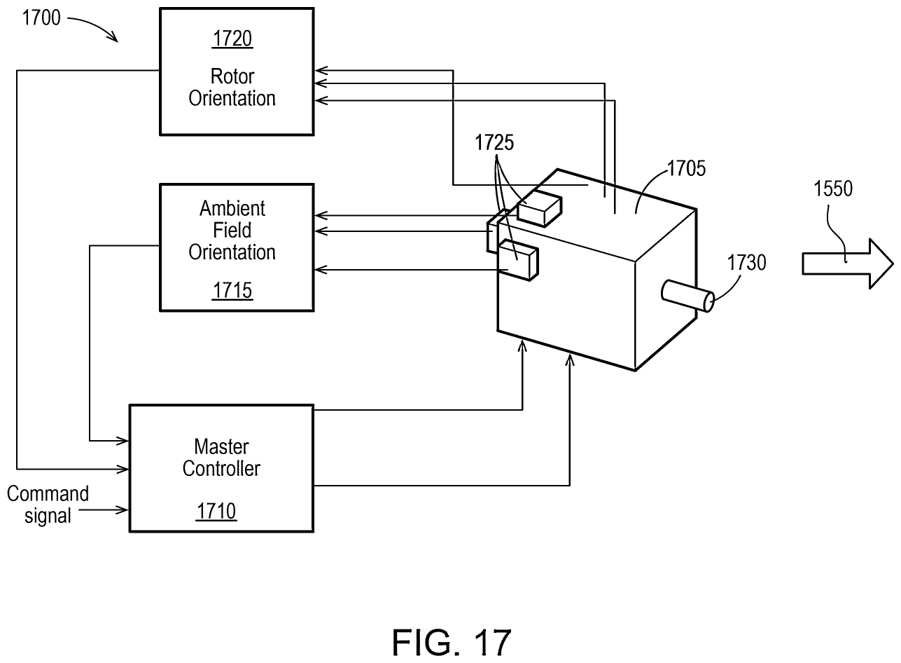

FIG. 17 depicts an exemplary h-AMFM system 1700. In this example, the h-AMFM system 1700 includes a h-AMFM 1705. In some implementations, the h-AMFM system 1700 may include electrical commutation schemes using slip rings to transfer power to the rotor (e.g., the coreless rotor 1605). For example, the h-AMFM 1705 may be operably coupled to a master controller 1710 to receive control signals. As shown, the master controller 1710 may be configured to operate the h-AMFM 1705 in four operation configurations. In some implementations, the h-AMFM system 1700 may include mechanical commutation schemes using brushes and a mechanical commutator to transfer power to the rotor (e.g., the coreless rotor 1605).

As shown, the h-AMFM system 1700 includes an ambient field orientation module 1715 and a rotor orientation module 1720. For example, the ambient field orientation module 1715 may receive signals from the Hall Effect sensors 1725 to determine an intensity vector of an ambient magnetic field 1550. For example, the rotor orientation module 1720 may receive signals from the Hall Effect sensors 1725 to determine whether the ambient magnetic field is optimally oriented with respect to the rotors of the AMFM to produce a torque at a motor output shaft 1730. For example, when the ambient magnetic field (MRI field) is less than a maximum field that can be produced by an auxiliary stator winding, the master controller 1710 may control to increase currents flow to auxiliary motor stator windings to generate a predominant stator field (e.g., the auxiliary stator field 1545).

When, for example, the ambient electromagnetic magnetic field is greater than a maximum field that can be produced by auxiliary stator winding, and the ambient field is optimally oriented with rotor producing torque. In this scenario, the ambient magnetic field 1550 from the MRI scanner contributes most significantly to motor torque output. For example, the master controller 1710 may reduce the current flowing to the auxiliary stator winding 1525.

When, for example, the ambient magnetic field is greater than a maximum field that can be produced by auxiliary stator winding, and the ambient field is not optimally oriented with rotor producing torque. In this scenario, the master controller 1710 may control the current in the auxiliary stator windings to produce a stator field that produces the optimal torque output.

When, the h-AMFM 1705 is in an MRI fringe field, for example, the ambient electromagnetic field may be close to a maximum field that can be produced by the auxiliary stator winding. For example, the auxiliary stator field 1545 may be controlled so that the combined effect of the ambient magnetic field 1550 and the auxiliary fields 1545 result in an output torque, specified by the command signal, on the rotor.

Although various embodiments have been described with reference to the figures, other embodiments are possible.

In some embodiments, an AMFM may include a sensor-less control strategy. Some embodiments may, for example, include a shaft encoder. As an illustrative example, a shaft encoder may be used in an (otherwise) sensorless control to provide feedback information for the motor controller.

The motor configurations described above can be converted to a servomotor using MRI-compatible encoders to measure relative or absolute position of output shaft and a closed loop controller. Motor shielding using conductive housing, shielded cables, motor shaft made from low electrical conductivity materials, and/or cable traps can be used to reduce RF noise (e.g., below a predetermined threshold acceptable for use in an MRI).

Although an exemplary system has been described with reference to FIGS. 1A-1B, other implementations may be deployed in other industrial, scientific, medical, commercial, and/or residential applications. One such illustrative application is a diagnostic application in which an actuator provides mechanical excitations to the body so that the tissue stiffness can be quantified with concurrent imaging with MRI.

Although various embodiments have been described with respect to an ambient electromagnetic field and/or an ambient magnetic field, other embodiments are possible. For example, an ambient magnetic field may be generated as an electromagnetic field. In some implementations, by way of example and not limitation, embodiments using an ambient electromagnetic field may be configured to use an ambient magnetic field. In some implementations, an ambient field may be external to the rotor and/or not specifically designed for and/or controlled by a motor controller. For example, in some implementations, an ambient field may not be mechanically linked to a rotor (e.g., the rotor and the ambient field may be orientable independently of each other during operation of the rotor). In some implementations, by way of example and not limitation, a strong ambient magnetic field may be at least an order of magnitude greater than the earth's magnetic field. In some implementations, for example, a strong ambient magnetic field may be generated by a device (e.g., MRI).

Computer program products may contain a set of instructions that, when executed by a processor device, cause the processor to perform prescribed functions. These functions may be performed in conjunction with controlled devices in operable communication with the processor. Computer program products, which may include software, may be stored in a data store tangibly embedded on a storage medium, such as an electronic, magnetic, or rotating storage device, and may be fixed or removable (e.g., hard disk, floppy disk, thumb drive, CD, DVD).

Some systems may be implemented as a computer system that can be used with various implementations. For example, various implementations may include digital circuitry, analog circuitry, computer hardware, firmware, software, or combinations thereof. Apparatus can be implemented in a computer program product tangibly embodied in an information carrier, e.g., in a machine-readable storage device, for execution by a programmable processor; and methods can be performed by a programmable processor executing a program of instructions to perform functions of various embodiments by operating on input data and generating an output. Various embodiments can be implemented advantageously in one or more computer programs that are executable on a programmable system including at least one programmable processor coupled to receive data and instructions from, and to transmit data and instructions to, a data storage system, at least one input device, and/or at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, which may include a single processor or one of multiple processors of any kind of computer. Generally, a processor will receive instructions and data from a read-only memory or a random-access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks, such as internal hard disks and removable disks; magneto-optical disks; and CD-ROM and DVD-ROM disks. The processor and the memory can be supplemented by, or incorporated in, ASICs (application-specific integrated circuits).

In some implementations, each system may be programmed with the same or similar information and/or initialized with substantially identical information stored in volatile and/or non-volatile memory. For example, one data interface may be configured to perform auto configuration, auto download, and/or auto update functions when coupled to an appropriate host device, such as a desktop computer or a server.

In some implementations, one or more user-interface features may be custom configured to perform specific functions. Various embodiments may be implemented in a computer system that includes a graphical user interface and/or an Internet browser. To provide for interaction with a user, some implementations may be implemented on a computer having a display device. The display device may, for example, include an LED (light-emitting diode) display. In some implementations, a display device may, for example, include a CRT (cathode ray tube). In some implementations, a display device may include, for example, an LCD (liquid crystal display). A display device (e.g., monitor) may, for example, be used for displaying information to the user. Some implementations may, for example, include a keyboard and/or pointing device (e.g., mouse, trackpad, trackball, joystick), such as by which the user can provide input to the computer.

In various implementations, the system may communicate using suitable communication methods, equipment, and techniques. For example, the system may communicate with compatible devices (e.g., devices capable of transferring data to and/or from the system) using point-to-point communication in which a message is transported directly from the source to the receiver over a dedicated physical link (e.g., fiber optic link, point-to-point wiring, daisy-chain). The components of the system may exchange information by any form or medium of analog or digital data communication, including packet-based messages on a communication network. Examples of communication networks include, e.g., a LAN (local area network), a WAN (wide area network), MAN (metropolitan area network), wireless and/or optical networks, the computers and networks forming the Internet, or some combination thereof. Other implementations may transport messages by broadcasting to all or substantially all devices that are coupled together by a communication network, for example, by using omni-directional radio frequency (RF) signals. Still other implementations may transport messages characterized by high directivity, such as RF signals transmitted using directional (i.e., narrow beam) antennas or infrared signals that may optionally be used with focusing optics. Still other implementations are possible using appropriate interfaces and protocols such as, by way of example and not intended to be limiting, USB 2.0, Firewire, ATA/IDE, RS-232, RS-422, RS-485, 802.11 a/b/g, Wi-Fi, Ethernet, IrDA, FDDI (fiber distributed data interface), token-ring networks, multiplexing techniques based on frequency, time, or code division, or some combination thereof. Some implementations may optionally incorporate features such as error checking and correction (ECC) for data integrity, or security measures, such as encryption (e.g., WEP) and password protection.

Various examples of modules may be implemented using circuitry, including various electronic hardware. By way of example and not limitation, the hardware may include transistors, resistors, capacitors, switches, integrated circuits, other modules, or some combination thereof. In various examples, the modules may include analog logic, digital logic, discrete components, traces and/or memory circuits fabricated on a silicon substrate including various integrated circuits (e.g., FPGAs, ASICs), or some combination thereof. In some embodiments, the module(s) may involve execution of preprogrammed instructions, software executed by a processor, or some combination thereof. For example, various modules may involve both hardware and software.

In an illustrative aspect, an actuator motor may, for example, include: a rotation unit (120) including at least one rotor (140), each of the at least one rotor may, for example, be configured to rotate about a corresponding rotation axis (141); and, a mechanical output shaft (150) mechanically coupled to the at least one rotor, wherein the mechanical output shaft extends along a longitudinal axis (151) that intersects at least one of the corresponding rotation axis, wherein the at least one rotor is configured to be selectively rotated by an ambient magnetic field (115) in response to a selectively applied electrical current through the at least one rotor, such that the at least one rotor is induced by the ambient magnetic field to generate an output torque at the mechanical output shaft about the longitudinal axis.

The mechanical output shaft may, for example, be coupled to the at least one rotor through a power mixing module. The power mixing module may, for example, be configured to generate the output torque by combining rotational torque induced at each of the at least one rotor. The power mixing module may, for example, include two miter gears mechanically coupling the at least one rotor to the motor output shaft.

The at least one rotor may, for example, include at least one rotor armature, wherein each of the at least one rotor armature includes at least one coil loop.

The actuator motor may, for example, include a controller operably coupled to the rotation unit. The controller may, for example, selectively control a rotor current flowing through electric windings on the at least one rotor based on at least a direction vector of the ambient electromagnetic field relative to the at least one rotor, such that the output torque generated at the mechanical output shaft is selectively controlled.

The actuator motor may, for example, include multiple Hall Effect sensors configured to generate a signal as a function of the direction vector of the ambient magnetic field. The controller may, for example, control the rotor current based on the signal generated by the Hall Effect sensors.

The actuator motor may, for example, include an MRI compatible encoder configured to provide position feedback information to the controller, such that the controller controls the actuator motor to produce a target angular position of the mechanical output shaft based on the position feedback information.

The controller may, for example, be enclosed in a conducting enclosure, such that electromagnetic noise generated from the controller is reduced.

The rotation unit may, for example, be enclosed in a conducting enclosure, such that radiated electromagnetic noise generated at each of the at least one rotor is reduced. The motor shaft may, for example, be made from low-conductivity material, such that electromagnetic noise radiated from the conducting enclosure is reduced.

The rotation unit may, for example, include multiple rotors, wherein each of the rotors rotates about intersecting axes of rotations. The rotation unit may, for example, include two rotors, wherein the axes of rotations of the two rotors are separated by 90°. The rotation unit may, for example, include three rotors, wherein the axes of rotations of the three rotors are separated by 60°.

The actuator motor may, for example, include an auxiliary stator, wherein the auxiliary stator includes motor stator windings configured to selectively generate an auxiliary magnetic field such that, when the output torque induced by an intensity vector of the ambient electromagnetic field is below a predetermined threshold, the rotation unit is selectively rotated by the auxiliary magnetic field generated by the auxiliary stator and electrical current in one or more rotor windings.

The actuator motor may, for example, include a stator current controller, wherein the stator current controller selectively controls a stator current flowing in the motor stator windings based on a command signal, an ambient field orientation, and a rotor rotation axis orientation.

The actuator motor may, for example, include an electric power transfer device configured to transfer electrical power to the rotor windings based on an orientation of brushes relative to at least one electrical conducting segment of a mechanical commutator. The at least one electrical conducting segment may, for example, rotate with a corresponding rotor of the at least one rotor. The at least one electrical conducting segment may, for example, include multiple separated segments. Rotor windings of the at least one rotor may, for example, be electrically connected to the at least one electrical conducting segment. A geometric orientation of the at least one electrical conducting segments with respect to the brushes, rotor armature windings, and ambient magnetic field direction may, for example, be configured such that electric currents supplied to the rotor armature are distributed to generate mechanical forces on the rotor that result in mechanical output of the rotor.

The actuator motor may, for example, include an electric power transfer device including brushes and continuous conductive contacts that rotate with a corresponding rotor of the at least one rotor. The continuous conductive contacts may, for example, continually electrically connect stationary electrical terminals to a rotating electrical component. The rotating electrical component may, for example, include armature windings. The rotating electrical component may, for example, include a sensor on the corresponding rotor.

The actuator motor may, for example, include switching circuitry and a controller configured to provide the selectively applied electrical current to the armature of the corresponding rotor to generate a target rotor output. The selectively applied electrical current is generated based on an orientation of the corresponding rotor in the ambient magnetic field. The selectively applied electrical current may, for example, be generated as a function of rotor position relative to the ambient magnetic field. The selectively applied electrical current may, for example, be generated as a function of estimated rotor position. The selectively applied electrical current may, for example, be generated as a function of measured rotor position.

The actuator motor may, for example, include a logic circuit and switches to selectively control current to the winding of the at least one rotor to produce the desired mechanical output.

The at least one rotor may, for example, be configured to actuate an end effector on a robot operable in a magnetic resonance imaging field in multiple degrees of freedom.

In an illustrative aspect, an actuator motor may, for example, include: a rotation unit (120) including multiple rotors (130, 140), each of the rotors is configured to independently rotate about multiple rotation axes (131, 141), wherein each of the rotors is configured to be selectively rotated in response to a selectively applied electrical current through at least one of the rotors, the selectively applied electrical current being based on a direction vector of an ambient magnetic field (115); and, a mechanical output shaft (150) mechanically coupled to the rotors, wherein the mechanical output shaft extends along a longitudinal axis (151) that intersects at least one of the rotation axes, wherein at least two of the rotation axes intersects with each other, such that: a torque constant (kT) at the mechanical output shaft is maintained within a predetermined range when the rotation unit changes in an orientation with respect to a direction of the ambient magnetic field. The axes of rotations of the rotors may, for example, be separated by 90°.

The rotation unit may, for example, include three rotors, wherein the axes of rotations of the three rotors are separated by 60°.

The mechanical output shaft may, for example, be coupled to the rotors through a power mixing module, wherein the power mixing module is configured to generate the output torque by combining rotational torque induced at each of the rotors. The power mixing module may, for example, include a gear coupled to each of the rotors, configured to translate a torque induced at each of the rotors mechanically to the motor output shaft.

Each of the rotors may, for example, include at least one rotor armature, wherein each of the at least one rotor armature includes at least one coil loop.

The actuator motor may, for example, include a controller operably coupled to the rotation unit, wherein the controller selectively controls a rotor current flowing through electric windings on the rotation unit based on at least a direction vector of the ambient magnetic field, such that the output torque generated at the mechanical output shaft is selectively controlled.

The actuator motor may, for example, include multiple Hall Effect sensors configured to generate a signal as a function of the direction vector of the ambient magnetic field, wherein the controller controls the rotor current based on the signal generated by the Hall Effect sensors.

The actuator motor may, for example, include an MRI compatible encoder configured to provide position feedback information to the controller, such that the controller controls the actuator motor to produce a target angular position of the mechanical output shaft based on the position feedback information. The controller may, for example, be enclosed in a conducting enclosure, such that electromagnetic noise generated from the controller is reduced.

The rotation unit may, for example, be enclosed in a conducting enclosure, such that radiated electromagnetic noise generated at the rotation unit is reduced.

The actuator motor may, for example, include an electric power transfer device configured to transfer electrical power to the rotor windings based on an orientation of brushes relative to at least one electrical conducting segment of a mechanical commutator. The at least one electrical conducting segment may, for example, rotate with a corresponding rotor of the at least one rotor. The at least one electrical conducting segment may, for example, include multiple separated segments. Rotor windings of the at least one rotor may, for example, be electrically connected to the at least one electrical conducting segment. A geometric orientation of the at least one electrical conducting segment with respect to the brushes, rotor armature windings, and ambient magnetic field direction may, for example, be configured such that electric currents supplied to the rotor armature are distributed to generate mechanical forces on the rotor that result in mechanical output of the rotor.

The actuator motor may, for example, include an electric power transfer device including brushes and continuous conductive contacts that rotate with a corresponding rotor of the at least one rotor. The continuous conductive contacts may, for example, continually electrically connect stationary electrical terminals to a rotating electrical component. The rotating electrical component may, for example, include armature windings. The rotating electrical component may, for example, include a sensor on the corresponding rotor.

The actuator motor may, for example, include switching circuitry and a controller configured to provide the selectively applied electrical current to the armature of the corresponding rotor such to generate a target rotor output. The selectively applied electrical current may, for example, be generated based on an orientation of the corresponding rotor in the ambient magnetic field. The selectively applied electrical current may, for example, be generated as a function of rotor position relative to the ambient magnetic field. The selectively applied electrical current may, for example, be generated as a function of estimated rotor position. The selectively applied electrical current may, for example, be generated as a function of measured rotor position.

The actuator motor may, for example, include a logic circuit and switches to selectively control current to the winding of the at least one rotor to produce the desired mechanical output.

The at least one rotor may, for example, be configured to actuate an end effector on a robot operable in an magnetic resonance imaging field in multiple degrees of freedom.

In an illustrative aspect, an actuator motor may, for example, include: a rotation unit (120) including at least one rotor (130), each of the at least one rotor being configured to rotate about a corresponding rotation axis (131); and, at least one auxiliary stator (158), wherein the at least one auxiliary stator includes motor stator windings (1525) configured to selectively generate an auxiliary magnetic field (1545) to induce rotation at the at least one rotor; and, a mechanical output shaft (150) mechanically coupled to the at least one rotor, wherein the at least one rotor is configured to be selectively rotated by an ambient magnetic field (115) in response to a selectively applied electrical current through the at least one rotor, such that, the at least one rotor is induced by the ambient magnetic field to generate an output torque at the mechanical output shaft about the longitudinal axis, and, when the output torque induced by the ambient magnetic field is below a predetermined threshold, the at least one rotor and the mechanical output shaft are selectively rotated at least by the auxiliary magnetic field generated by the auxiliary stator.

The mechanical output shaft may, for example, extend along a longitudinal axis (151) that intersects at least one of the corresponding rotation axes.

The actuator motor may, for example, include a stator current controller, wherein the stator current controller selectively controls a stator current flowing in the motor stator windings based on a command signal, an ambient field orientation, and a rotor orientation.

The mechanical output shaft may, for example, be coupled to the at least one rotor through a power mixing module, wherein the power mixing module is configured to generate the output torque by combining rotational torque induced at each of the at least one rotor. The power mixing module may, for example, include two miter gears mechanically coupling the at least one rotor to the motor output shaft.

The at least one rotor may, for example, include at least one rotor armature, wherein each of the at least one rotor armature includes at least one coil loop.

The actuator motor may, for example, include a controller operably coupled to the rotation unit, wherein the controller selectively controls a rotor current flowing through electric windings on the at least one rotor based on at least the direction vector of the ambient magnetic field, such that the output torque generated at the mechanical output shaft is selectively controlled. The actuator motor may, for example, include Hall Effect sensors configured to generate a signal as a function of the direction vector of the ambient magnetic field, wherein the controller controls the rotor current based on the signal generated by the Hall Effect sensors. The controller may, for example, control the stator current based on the signal generated by the Hall Effect sensors. The actuator motor may, for example, include an MRI compatible encoder configured to provide position feedback information to the controller, such that the controller controls the actuator motor to produce a target angular position of the mechanical output shaft based on the position feedback information. The controller may, for example, be enclosed in a conducting enclosure, such that electromagnetic noise generated from the controller is reduced.

The rotation unit may, for example, be enclosed in a conducting enclosure, such that radiated electromagnetic noise generated at each of the at least one rotor is reduced. The motor shaft may, for example, be made from low-conductivity material, such that electromagnetic noise radiated from the conducting enclosure is reduced.

The rotation unit may, for example, include a coreless rotor.

The actuator motor may, for example, include an electric power transfer device configured to transfer electrical power to the rotor windings based on an orientation of brushes relative to at least one electrical conducting segment of a mechanical commutator. The at least one electrical conducting segment may, for example, rotate with a corresponding rotor of the at least one rotor. The at least one electrical conducting segment may, for example, include multiple separated segments. Rotor windings of the at least one rotor may, for example, be electrically connected to the at least one electrical conducting segment. A geometric orientation of the at least one electrical conducting segments with respect to the brushes, rotor armature windings, auxiliary stator windings, and ambient magnetic field direction may, for example, be configured such that electric currents supplied to the rotor armature are distributed to generate mechanical forces on the rotor that result in mechanical output of the rotor.

The actuator motor may, for example, include an electric power transfer device including brushes and continuous conductive contacts that rotate with a corresponding rotor of the at least one rotor. The continuous conductive contacts may, for example, continually electrically connect stationary electrical terminals to a rotating electrical component. The rotating electrical component may, for example, include armature windings. The rotating electrical component may, for example, include a sensor on the corresponding rotor.

The actuator motor may, for example, include switching circuitry and a controller configured to provide the selectively applied electrical current to the armature of the corresponding rotor to generate a target rotor output.

The selectively applied electrical current may, for example, be generated based on an orientation of the corresponding rotor in the ambient magnetic field. The selectively applied electrical current is generated as a function of rotor position relative to the ambient magnetic field. The selectively applied electrical current may, for example, be generated as a function of estimated rotor position. The selectively applied electrical current may, for example, be generated as a function of measured rotor position.

The actuator motor may, for example, include a logic circuit and switches to selectively control current to the winding of the at least one rotor to produce the desired mechanical output. The selectively applied electrical current may, for example, be generated based on an orientation of the corresponding rotor in the auxiliary magnetic field. The selectively applied electrical current may, for example, be generated as a function of rotor position relative to the auxiliary magnetic field. The selectively applied electrical current may, for example, be generated as a function of estimated rotor position. The selectively applied electrical current may, for example, be generated as a function of measured rotor position.

The at least one rotor may, for example, be configured to actuate an end effector on a robot operable in a magnetic resonance imaging field in multiple degrees of freedom.

The at least one rotor may, for example, be configured to actuate a mechanical system including a degree of freedom on a robot.

A number of implementations have been described. Nevertheless, it will be understood that various modifications may, for example, be made. For example, advantageous results may, for example, be achieved if the steps of the disclosed techniques were performed in a different sequence, or if components of the disclosed systems were combined in a different manner, or if the components were supplemented with other components. Accordingly, other implementations are contemplated within the scope of the following claims.

1. An actuator motor comprising:
a rotation unit (120) comprising at least one rotor (140), each of the at least one rotor is configured to rotate about a corresponding rotation axis (141) and configured to receive a selectively applied electrical current by a power source; and,
a mechanical output shaft (150) mechanically coupled to the at least one rotor, wherein the mechanical output shaft extends along a longitudinal axis (151) that intersects at least one of the corresponding rotation axis, wherein the at least one rotor is configured to be selectively rotated by an ambient magnetic field (115) in response to the selectively applied electrical current through the at least one rotor, wherein the ambient magnetic field is independently generated from an external magnetic source separated from the actuator motor, such that the at least one rotor is induced by the ambient magnetic field to generate an output torque at the mechanical output shaft about the longitudinal axis.

2. The actuator motor of claim 1, wherein the mechanical output shaft is coupled to the at least one rotor through a power mixing module, wherein the power mixing module is configured to generate the output torque by combining rotational torque induced at each of the at least one rotor.

3-4. (canceled)

5. The actuator motor of claim 1, further comprising a controller operably coupled to the rotation unit, wherein the controller selectively controls a rotor current flowing through electric windings on the at least one rotor based on at least a direction vector of the ambient electromagnetic field relative to the at least one rotor, such that the output torque generated at the mechanical output shaft is selectively controlled.

6. The actuator motor of claim 5, further comprising a plurality of Hall Effect sensors configured to generate a signal as a function of the direction vector of the ambient magnetic field, wherein the controller controls the rotor current based on the signal generated by the plurality of Hall Effect sensors.

7. The actuator motor of claim 5, further comprising an MRI compatible encoder configured to provide position feedback information to the controller, such that the controller controls the actuator motor to produce a target angular position of the mechanical output shaft based on the position feedback information.

8-13. (canceled)

14. The actuator motor of claim 1, further comprising an auxiliary stator, wherein the auxiliary stator comprises motor stator windings configured to selectively generate an auxiliary magnetic field such that, when the output torque induced by an intensity vector of the ambient electromagnetic field is below a predetermined threshold, the rotation unit is selectively rotated by the auxiliary magnetic field generated by the auxiliary stator and electrical current in one or more rotor windings.

15. (canceled)

16. The actuator motor of claim 1, further comprising an electric power transfer device configured to transfer electrical power to rotor windings based on an orientation of brushes relative to at least one electrical conducting segment of a mechanical commutator.

17-20. (canceled)

21. The actuator motor of claim 1, further comprising an electric power transfer device comprising brushes and continuous conductive contacts that rotate with a corresponding rotor of the at least one rotor.

22-30. (canceled)

31. The actuator motor of claim 1, wherein the at least one rotor is configured to actuate an end effector on a robot operable in a magnetic resonance imaging field in multiple degrees of freedom.

32. An actuator motor comprising:
a rotation unit (120) comprising a plurality of rotors (130, 140), each of the plurality of rotors being configured to independently rotate about a plurality of rotation axes (131, 141), wherein each of the plurality of rotors is configured to be selectively rotated in response to a selectively applied electrical current through at least one of the plurality of rotors, the selectively applied electrical current being generated based on a direction vector of an ambient magnetic field (115); and,
a mechanical output shaft (150) mechanically coupled to the plurality of rotors, wherein the mechanical output shaft extends along a longitudinal axis (151) that intersects at least one of the plurality of rotation axes, wherein at least two of the plurality of rotation axes intersects with each other, such that:
a torque constant (kT) at the mechanical output shaft is maintained within a predetermined range when the rotation unit changes in an orientation with respect to a direction of the ambient magnetic field.

33-34. (canceled)

35. The actuator motor of claim 32, wherein the mechanical output shaft is coupled to the plurality of rotors through a power mixing module, wherein the power mixing module is configured to generate an output torque by combining rotational torque induced at each of the plurality of rotors.

36-37. (canceled)

38. The actuator motor of claim 32, further comprising a controller operably coupled to the rotation unit, wherein the controller selectively controls a rotor current flowing through electric windings on the rotation unit based on at least the direction vector of the ambient magnetic field, such that an output torque generated at the mechanical output shaft is selectively controlled.

39-42. (canceled)

43. The actuator motor of claim 32, further comprising an electric power transfer device configured to transfer electrical power to rotor windings of the plurality of rotors based on an orientation of brushes relative to at least one electrical conducting segment of a mechanical commutator.

44-58. (canceled)

59. An actuator motor comprising:
a rotation unit (120) comprising
at least one rotor (130), each of the at least one rotor is configured to rotate about a corresponding rotation axis (131) and configured to receive a selectively applied electrical current from a power source;
at least one auxiliary stator (158), wherein the at least one auxiliary stator comprises motor stator windings (1525) configured to selectively generate an auxiliary magnetic field (1545) to induce rotation at the at least one rotor; and,
a mechanical output shaft (150) mechanically coupled to the at least one rotor, wherein the at least one rotor is configured to be selectively rotated by an ambient magnetic field (115) in response to the selectively applied electrical current through the at least one rotor, such that,
the at least one rotor is induced by the ambient magnetic field to generate an output torque at the mechanical output shaft about a longitudinal axis (151) of the mechanical output shaft, and,
when the output torque induced by the ambient magnetic field is below a predetermined threshold, the at least one rotor and the mechanical output shaft are selectively rotated at least by the auxiliary magnetic field generated by the at least one auxiliary stator.

60. The actuator motor of claim 59, wherein the rotation unit comprises a plurality of rotors configured to rotate about a plurality of rotation axes, wherein the longitudinal axis intersects at least one of the plurality of rotation axes, wherein at least two of the plurality of rotation axes intersects with each other, such that:
a torque constant (kT) at the mechanical output shaft is maintained within a predetermined range when the rotation unit changes in an orientation with respect to a direction of the ambient magnetic field.

61. The actuator motor of claim 59, further comprising a stator current controller, wherein the stator current controller selectively controls a stator current flowing in the motor stator windings based on a command signal, an ambient field orientation, and a rotor orientation.

62. The actuator motor of claim 59, wherein the mechanical output shaft is coupled to the at least one rotor through a power mixing module, wherein the power mixing module is configured to generate the output torque by combining rotational torque induced at each of the at least one rotor.

63-71. (canceled)

72. The actuator motor of claim 59, wherein the rotation unit comprises a coreless rotor.

73. The actuator motor of claim 59, further comprising an electric power transfer device configured to transfer electrical power to rotor windings based on an orientation of brushes relative to at least one electrical conducting segment of a mechanical commutator.

74-77. (canceled)

78. The actuator motor of claim 59, further comprising an electric power transfer device comprising brushes and continuous conductive contacts that rotate with a corresponding rotor of the at least one rotor.

79-93. (canceled)
